(19) United States
(12) Patent Application Publication
(10) Pub. No.: US 2023/0080313 A1
(43) Pub. Date: Mar. 16, 2023

(54) CHANNEL SELECTION FOR ADDRESSING CHRONIC CONDITIONS

(71) Applicant: CVS Pharmacy, Inc., Woonsocket, RI (US)

(72) Inventors: Eli Goldberg, Norfolk, MA (US); Lauren Lamonica, Ann Arbor, MI (US); Paul Raff, Pittsburgh, PA (US); Michael Turner, Wakefield, MA (US); Eugenio Zuccarelli, New York, NY (US); Lingli Duan, Boston, MA (US)

(21) Appl. No.: 17/902,613

(22) Filed: Sep. 2, 2022

Related U.S. Application Data
(60) Provisional application No. 63/240,744, filed on Sep. 3, 2021.

(51) Int. Cl. G16H 50/70; G16H 10/60

(52) U.S. Cl. CPC G16H 50/70; G16H 10/60

(57) ABSTRACT

A system, method, and apparatus for managing care gaps associated with a member are provided. The method may include determining a plurality of current gaps-in-care for the member, where each of the plurality of current gaps-in-care for the member is defined as a difference between a current health-related behavior of the member and a guideline behavior for the member; determining for each of the plurality of current gaps-in-care for the member a potential expected value to be captured by reducing the difference; selecting a current gap-in-care from among the plurality of gaps-in-care as a gap-to-address, where the current gap-in-care is selected based on having a higher potential value as compared to at least one other gap-in-care among the plurality of gaps-in-care; and communicating one or more actions for the member to take in connection with at least partially closing the gap-in-care.

### CROSS REFERENCE TO RELATED APPLICATIONS

This application claims the benefit of and priority, under 35 U.S.C. § 119(e), to U.S. Provisional Application No. 63/240,744, filed on Sep. 3, 2021, and entitled “AI Based Methods And Systems For Tracking Chronic Conditions,” the entire disclosure of which is hereby incorporated herein by reference, in its entirety, for all that it teaches and for all purposes.

### FIELD OF THE DISCLOSURE

The disclosure relates to systems and methods for identification, tracking, and treatment of chronic diseases (e.g., Type 2 diabetes) based on a modeling analysis of gaps in care of a patient.

### BACKGROUND

Pharmaceutical manufacturers, healthcare providers, pharmaceutical distributors, and other entities involved in the delivery of treatments to members are able to observe medical histories of their members. Healthcare providers routinely access treatment histories of patients under their care. Decision aids for managing gaps-in-care associated with a patient, for example, with respect to chronic diseases (e.g., Type 2 diabetes), are desired. The terms “gap-in-care,” “care gap,” and “gap” may be used interchangeably herein.

### SUMMARY

According to example aspects of the present disclosure, a machine learning model(s) (also referred to herein as an artificial intelligence (AI) model) may support observing member behavior and managing a care gap associated with a member. In some aspects, the machine learning model(s) may support channel selection for managing a care gap associated with a member. In some other aspects, the machine learning model(s) may support cyclical and/or continuous management of care gaps associated with a member. The terms “member” and “patient” be used interchangeably herein. A “care gap” or “gap-in-care” for a member may be ranked according to one or more criteria. As care gaps are ranked, it may become possible to select, pick, or choose which care gap(s) among a plurality of care gaps provides a highest probability of success as measured by a number of different factors (e.g., which care gap(s), if closed or reduced, may provide a maximum positive impact for the member). It may also be possible to select, pick, or choose which channel among a plurality of channels may be most likely to close a gap for a member and which gaps among a plurality of gaps may provide a maximum positive impact for that member.

In one aspect, a system, method, and apparatus for observing member behavior and managing a care gap associated with the member are provided that include: determining a current gap-in-care for the member by: receiving an electronic record associated with the member that describes a current health-related behavior of the member; receiving guideline behavior for the member supported by a professional clinical recommendation; and determining a difference between the current health-related behavior of the member and the guideline behavior for the member, where the difference defines, at least in part, the current gap-in-care for the member. Some examples of the system, method, and apparatus may include determining, for the current gap-in-care for the member, one or more actions for the member that, if followed, are capable of at least partially closing the current gap-in-care within a clinically-defined period of time for the member; determining, for the one or more actions, an impact associated with at least partially closing the current gap-in-care for the member according to the one or more actions; and providing a communication to the member that describes the one or more actions for the member.

In another aspect, a system, method, and apparatus for channel selection for managing a care gap associated with a member are provided that include: determining a current gap-in-care for the member, where the current gap-in-care for the member may be defined as a difference between a current health-related behavior of the member and a guideline behavior for the member; determining, for a channel, a probability of at least partially closing the current gap-in-care within a clinically-defined period of time for the member; determining, for the channel(s), a value associated with at least partially closing the current gap-in-care for the member; selecting the channel (e.g., email, direct mail, short message service (SMS), etc.) from among a plurality of candidate channels based on a combination of the determined probability and the determined value associated with at least partially closing the current gap-in-care for the member; and providing a communication (or multiple communications) to the member and/or provider via the selected channel that describes one or more actions for the member to take in connection with at least partially closing the current gap-in-care for the member and an impact associated with taking the one or more actions.

In another aspect, a system, method, and apparatus for managing care gaps associated with a member are provided that include: determining a plurality of current gaps-in-care for the member, where each of the plurality of current gaps-in-care for the member may be defined as a difference between a current health-related behavior of the member and a guideline behavior for the member; determining for each of the plurality of current gaps-in-care for the member a potential expected value to be captured by reducing the difference; selecting a current gap-in-care from among the plurality of gaps-in-care as a gap-to-address; and communicating one or more actions for the member to take in connection with at least partially closing the gap-in-care. In some aspects, the current gap-in-care may be selected based on having a higher potential value as compared to at least one other gap-in-care among the plurality of gaps-in-care. In some aspects, the one or more actions are communicated to the member via a channel selected to provide a highest probability of achieving the higher potential value.

All examples and features mentioned above can be combined in any technically possible way.

### BRIEF DESCRIPTION OF THE DRAWINGS

The present disclosure is described in conjunction with the appended figures, which are not necessarily drawn to scale:

FIG. 1 illustrates an example of a system that supports artificial intelligence (AI) based methods and systems for tracking chronic conditions in accordance with aspects of the present disclosure.

FIG. 2 illustrates an example of machine learning and game theory-based approaches in accordance with aspects of the present disclosure.

FIG. 3 illustrates an example of actions a member may take in association with closing a gap-in-care in accordance with aspects of the present disclosure.

FIG. 4 illustrates an example block diagram that supports gap-in-care valuation, prioritization, and channel selection in accordance with aspects of the present disclosure.

FIG. 5 illustrates an example operational flow that supports tracking chronic conditions in accordance with aspects of the present disclosure.

FIGS. 6 through 9 illustrate example process flows that support tracking chronic conditions in accordance with aspects of the present disclosure.

FIG. 10 illustrates an example operational flow in accordance with aspects of the present disclosure.

### DETAILED DESCRIPTION

Before any examples of the disclosure are explained in detail, it is to be understood that the disclosure is not limited in its application to the details of construction and the arrangement of components set forth in the following description or illustrated in the following drawings. The disclosure is capable of other configurations and of being practiced or of being carried out in various ways. Also, it is to be understood that the phraseology and terminology used herein is for the purpose of description and should not be regarded as limiting.

While various examples of addressing care gaps of a member or patient will be described in connection with a member or patient having diabetes, it should be appreciated that the disclosure is not so limited. For instance, it is contemplated that examples of the present disclosure can be applied to manage care gaps of many different types for members or patients having any number of different conditions that could benefit from care or treatment adherence. In other words, the framework described herein for managing care gaps can be leveraged to support care management opportunities and/or manage any type or number of different medical conditions. Examples of such medical conditions that can be addressed or improved with the framework described herein include, without limitation, cardiac conditions, heightened cholesterol, heightened blood pressure, hypertension, post-operative conditions, pre-operative conditions, cancer and other chronic conditions, infertility, chronic pain, broken bones, torn ligaments, torn muscles, etc.

Diabetes mellitus is a complex, progressive chronic condition in which the body's ability to produce or respond to the hormone insulin is impaired. Such impairment may result in abnormal metabolism of carbohydrates and elevated levels of glucose in the blood and urine of a patient.

Some approaches to achieving optimal diabetes control are hindered by poor medication adherence, high treatment cost, clinical inertia, and inadequate access to glucose testing and preventative screening. For example, some ‘one-size-fits-all’ approaches to diabetes management are unlikely to address a most important/highly ranked/highest ranked (e.g., based on return-on-investment (ROI), cost impact, clinical impact, etc.) gap-in-care that prevents a member from controlling their blood glucose levels. In some cases, for each member, the single next best action expected to yield the highest impact on diabetes control is highly dependent on features of the member (e.g., comorbidity profiles, treatment adherences, lifestyle factors, medical history, social determinants of health, etc.).

Some techniques for managing diabetes are further complicated by the growing rate of new diabetes diagnoses in comparison to the number of practicing specialists and providers. The rise in diabetes diagnoses is complicated by a critical shortage in primary care providers and endocrinologists, and the ratio of members with diabetes to care providers is projected to continue to increase. Traditionally, diabetes management has resided within the purview of medical providers (e.g., clinicians). In some healthcare management systems, members now have a heightened responsibility to support systemic changes designed to keep an increased number of diabetes members healthy and to mitigate rising health care costs.

In some cases, personalized diabetes approaches have largely centered on micro-control of blood glucose through mobile applications and medical devices. While these applications may provide an effective means of connecting to members, such applications have not addressed the issue that not every treatment pathway works for every member. Additionally, while an emphasis on tight glucose control may be beneficial for some members (e.g., members having a number of diabetes-associated complications and comorbidities below a threshold), such an approach may present problems for other members (e.g., members having a number of diabetes-associated complications and comorbidities above the threshold).

Machine learning and deep learning-based approaches have built upon the abilities of clinicians to assess current complications and predict the future health conditions of a member. For example, some deep learning approaches are able to detect diabetic retinopathy from retinal fundus photographs of adults with diabetes with high specificity and sensitivity, offering a potential to improve screening in clinical settings. However, while some machine learning approaches may yield higher accuracy with respect to assessing current complications and predicting the future health conditions of a member, clinical applications of such these approaches have been scarce due to an inability of some systems to translate model findings to actionable member interventions. Approaches capable of offering a tangible implementation to actionable member interventions (e.g., a tangible implementation framework) are desired.

Accordingly, for example, among members with diabetes (e.g., Type 2 diabetes), some healthcare management systems are ineffective in determining and addressing the member-level impact of closing individual gaps in care. For example, some healthcare management systems may be ineffective in prioritizing gap closure for a given member (e.g., to optimize glucose control) because they are unaware of the methods or modalities by which that member may wish to communicate or be unaware of the impact of a gap in their care that may be outsized in comparison to its traditional clinical importance. Accordingly, for example, precision treatment of a medical condition (e.g., diabetes, cardiac conditions, heightened cholesterol, heightened blood pressure, hypertension, post-operative conditions, pre-operative conditions, cancer and other chronic conditions, infertility, chronic pain, broken bones, torn ligaments, torn muscles, etc.) may offer the potential to deliver more personalized clinical interventions.

According to example aspects of the present disclosure, machine learning and game theory combinatorial techniques are described which support identifying highly ranked influential clinical features and gaps in care in controlling and/or reducing hemoglobin A1c (HbA1c) for each individual member, driving intervention-action pairs to provide improved diabetes management. In some aspects, the techniques may include the application of a machine learning and game theory based model. The machine learning and game theory-based model may be generated and trained based on a feature set including insurance claims, pharmacy records, clinically-relevant biomarkers and laboratory results, healthcare utilization history, and gaps in care. In some aspects, the machine learning and game theory based model is provided which leverages Shapley values and is trained on a population (e.g., member set) of insured members with Type 2 diabetes. In an example, a healthcare management system described herein may implement the machine learning and game theory-based model to generate intervention-action pairs applicable to launching communication campaigns among members of a healthcare network.

The techniques described herein support identifying the prevalence of diabetes-related comorbidities, clinical characteristics, and demographic patterns in members with diabetes (e.g., Type 2 diabetes) within a health insurance provider system to design intervention-action pairs that can close the one or more highly ranked gaps in diabetes care management. For example, precision diabetes treatment techniques are described that are capable of enhancing the delivery of healthcare for diabetes members by driving targeted clinically-meaningful intervention-action pairs with the highest potential to influence estimated average glucose (also referred to herein A1c or eAG).

Example aspects of the present disclosure are described with reference to the following figures.

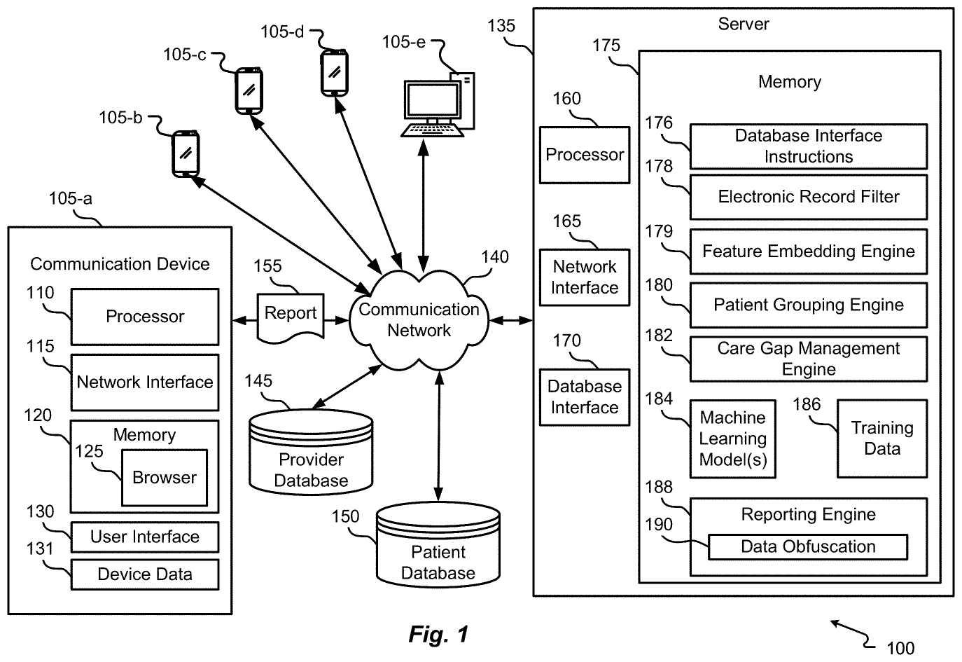

FIG. 1 illustrates an example of a system 100 that supports tracking chronic conditions (e.g., diabetes, blood pressure, chronic pain, etc.) in accordance with aspects of the present disclosure. The system 100, in some examples, may include one or more computing devices operating in cooperation with one another to observe member behavior and manage a care gap associated with a member. In some aspects, the system 100 may support channel selection for managing a care gap associated with a member. In some other aspects, the system 100 may support cyclical and/or continuous management of care gaps associated with a member. The system 100 may be, for example, a healthcare management system.

The components of the system 100 may be utilized to facilitate one, some, or all of the methods described herein or portions thereof without departing from the scope of the present disclosure. Furthermore, the servers described herein may include example components or instruction sets, and aspects of the present disclosure are not limited thereto. In an example, a server may be provided with all of the instruction sets and data depicted and described in the server of FIG. 1. Alternatively, or additionally, different servers or multiple servers may be provided with different instruction sets than those depicted in FIG. 1.

The system 100 may include communication devices 105 (e.g., communication device 105-a through communication device 105-e), a server 135, a communication network 140, a provider database 145, and a member database 150. The communication network 140 may facilitate machine-to-machine communications between any of the communication devices 105 (or multiple communication devices 105), the server 135, or one or more databases (e.g., a provider database 145, a member database 150). The communication network 140 may include any type of known communication medium or collection of communication media and may use any type of protocols to transport messages between endpoints. The communication network 140 may include wired communications technologies, wireless communications technologies, or any combination thereof.

The Internet is an example of the communication network 140 that constitutes an Internet Protocol (IP) network consisting of multiple computers, computing networks, and other communication devices located in multiple locations, and components in the communication network 140 (e.g., computers, computing networks, communication devices) may be connected through one or more telephone systems and other means. Other examples of the communication network 140 may include, without limitation, a standard Plain Old Telephone System (POTS), an Integrated Services Digital Network (ISDN), the Public Switched Telephone Network (PSTN), a Local Area Network (LAN), a Wide Area Network (WAN), a wireless LAN (WLAN), a Session Initiation Protocol (SIP) network, a Voice over Internet Protocol (VoIP) network, a cellular network, and any other type of packet-switched or circuit-switched network known in the art. In some cases, the communication network 140 may include of any combination of networks or network types. In some aspects, the communication network 140 may include any combination of communication mediums such as coaxial cable, copper cable/wire, fiber-optic cable, or antennas for communicating data (e.g., transmitting/receiving data).

A communication device 105 (e.g., communication device 105-a) may include a processor 110, a network interface 115, a computer memory 120, a user interface 130, and device data 131. In some examples, components of the communication device 105 (e.g., processor 110, network interface 115, computer memory 120, user interface 130) may communicate over a system bus (e.g., control busses, address busses, data busses) included in the communication device 105. In some cases, the communication device 105 may be referred to as a computing resource. The communication device 105 may establish one or more connections with the communication network 140 via the network interface 115. In some cases, the communication device 105 may transmit or receive packets to one or more other devices (e.g., another communication device 105, the server 135, the provider database 145, the provider database 150) via the communication network 140.

Non-limiting examples of the communication device 105 may include, for example, personal computing devices or mobile computing devices (e.g., laptop computers, mobile phones, smart phones, smart devices, wearable devices, tablets, etc.). In some examples, the communication device 105 may be operable by or carried by a human user. In some aspects, the communication device 105 may perform one or more operations autonomously or in combination with an input by the user.

The communication device 105 may support one or more operations or procedures associated with observing member behavior and managing a care gap associated with a member, channel selection for managing a care gap associated with a member, and cyclical and/or continuous management of care gaps associated with a member. For example, the communication device 105 may support communications between multiple entities such as a healthcare provider, a medical insurance provider, a pharmaceutical manufacturer, a pharmaceutical distributor, or combinations thereof. In some cases, the system 100 may include any number of communication devices 105, and each of the communication devices 105 may be associated with a respective entity.

The communication device 105 may render or output any combination of notifications, messages, menus, etc. based on data communications transmitted or received by the communication device 105 over the communication network 140. For example, the communication device 105 may receive one or more reports 155 (e.g., from the server 135) via the communication network 140. In some aspects, the communication device 105 may render a presentation (e.g., visually, audibly, using haptic feedback, etc.) of the report 155 via the user interface 130. The user interface 130 may include, for example, a display, an audio output device (e.g., a speaker, a headphone connector), or any combination thereof. In some aspects, the communication device 105 may render a presentation using one or more applications (e.g., a browser application 125) stored on the memory 120. In an example, the browser application 125 may be configured to receive the report 155 in an electronic format (e.g., in an electronic communication via the communication network 140) and present content of the report 155 via the user interface 130.

In some aspects, the report 155 may be a communication including one or more actions for a member that, if followed, are capable of at least partially closing a current gap-in-care within a clinically-defined period of time for the member. In some aspects, the server 135 may communicate the report 155 to a communication device 105 (e.g., communication device 105-a) of a member, a communication device 105 (e.g., communication device 105-b) of a healthcare provider, a communication device 105 (e.g., communication device 105-c) of an insurance provider, a communication device 105 (e.g., communication device 105-d) of a pharmacist, a communication device 105 (e.g., communication device 105-e) of team outreach personnel, or the like. Additionally, or alternatively, the server 135 may communicate a physical representation (e.g., a letter) of the report 155 to the member, a healthcare provider, an insurance provider, a pharmacist, team outreach personnel, or the like via a direct mail provider (e.g., postal service).

The provider database 145 and the member database 150 may include member electronic records (also referred to herein as a data records) stored therein. In some aspects, the electronic records may be accessible to a communication device 105 (e.g., operated by healthcare provider personnel, insurance provider personnel, a member, a pharmacist, etc.) and/or the server 135. In some aspects, a communication device 105 and/or the server 135 may receive and/or access the electronic records from the provider database 145 and the member database 150 (e.g., based on a set of permissions).

In some aspects, an electronic record associated with a member may include claims-based electronic data. For example, the electronic record may include electronic medical record (EMR) data. In another example, the claims-based electronic data may include data describing an insurance medical claim, pharmacy claim, and/or insurance claim made by the member and/or a medical provider. Accordingly, for example, the claims-based electronic data may come from providers or payers, and claims included in the claims-based electronic data may be of various types (e.g., medical, pharmacy, etc.).

In some other aspects, the electronic record associated with the member may include device data 131 obtained from a communication device 105 (e.g., communication device 105-a) associated with the member. For example, the device data 131 may include gyroscopic data, accelerometer data, beacon data, glucose readings, heart rate data, blood pressure data, blood oxygen data, temperature data, kinetics data, location data, motion data, a device identifier, and/or temporal data (e.g., a timestamp) measurable, trackable, and/or providable by the communication device 105 (or a device connected to the communication device 105) associated with the member.

In some aspects, the electronic record may include an image of the member. For example, the electronic record may include imaging data based on which the server 135 (e.g., the care gap management engine 182) may track targeted biomarkers. For example, the server 135 may track X-ray records of a member over time (e.g., in associated with assisting reduced healing times for a member). In some cases, the electronic record may include other types of diagnostic images such as magnetic resonance imaging (MM) scans, computed tomography scans (CT), ultrasound images, or the like.

In accordance with aspects of the present disclosure, the device data 131 may be provided continuously, semi-continuously, periodically, and/or based on a trigger condition by the communication device 105 (e.g., a smart watch, a wearable monitor, a self-reporting monitor such as a glucometer, a smartphone carried by a user, etc.) around monitored parameters such as heartbeat, blood pressure, etc. In some aspects, the device data 131 of a communication device 105 (e.g., communication device 105-a) may be referred to as “environmental data” associated with a user, which may be representative of aspects of environmental factors (e.g., lifestyle, socioeconomic factors, details about the environment, etc.) associated with a member.

Accordingly, for example, the electronic record may provide insurance claim information and/or generic behaviors of member behavior (e.g., behavior common to a set of members). In some cases, the device data 131 may include wearable-device data, glucose readings, heart rate, body temperature, “invisible” data (e.g., device related information associated with a member, such as Bluetooth beacon information), and/or self-reporting monitored data (e.g., provided by self-reporting glucometers such as continuous glucose monitors (CGMs) that report kinetics).

In some aspects, the electronic record may include genetic data associated with a member. In some other aspects, the electronic record may include notes/documentation that is recorded at a communication device 105 in a universal and/or systematic format (e.g., subjective, objective, assessment, and plan (SOAP) notes/documentation) among medical providers, insurers, etc. In some examples, the electronic record may include non-claim adjudicated diagnoses input at a communication device 105 (e.g., diagnoses that have not been evaluated by an insurance provider with respect to payment of benefits).

In some other aspects, the electronic records may be inclusive of aspects of a member's health history and health outlook. The electronic records may include a number of fields for storing different types of information to describe the member's health history and health outlook. As an example, the electronic records may include member identifier information such as, for example, name, address, member number, social security number, date of birth, etc. In some aspects, the electronic records may include treatment data such as, for example, member health history, member treatment history, lab test results (e.g., text-based, image-based, or both), pharmaceutical treatments and therapeutic treatments (e.g., indicated using predefined healthcare codes, treatment codes, or both), insurance claims history, healthcare provider information (e.g., doctors, therapists, etc. involved in providing healthcare services to the member), in-member information (e.g., whether treatment is associated with care), location information (e.g., associated with treatments or prescriptions provided to the member), family history (e.g., inclusive of medical data records associated with family members of the member, data links to the records, etc.), or any combination thereof. In some aspects, the electronic records may be stored or accessed according to one or more common field values (e.g., common parameters such as common healthcare provider, common location, common claims history, etc.).

In some aspects, the server 135 may receive the guideline behavior for the member supported by a professional clinical recommendation. For example, the server 135 may receive and/or access the guideline behavior from a communication device 105, the provider database 145, the member database 150, and/or another server 135. In some examples, the guideline behavior for the member supported by the professional clinical recommendation may include guidance based on at least one of medical history, demographics, social indices, biomarkers, behavior data, engagement data, historical gap-in-care data, and a machine learning model-derived output (e.g., a risk-based model probability derived by a machine learning model(s) 184 described herein). In some aspects, the guidance may be based on medical history, demographics, social indices, biomarkers, behavior data, engagement data, historical gap-in-care data, and/or machine learning model-derived output(s) that correspond to the member and/or other members.

In some aspects, the gap-in-care described herein may be defined by a difference between guideline behavior associated with what a member should be doing, as defined by clinical guidelines and expert clinical opinion (e.g., professional guidelines surrounding preventative screenings and close follow-up and monitoring with healthcare providers) and current health related behavior associated with what the member is actually doing, which may be defined by static or longitudinal observables in the medical history of the member and supporting data.

In some aspects, the provider database 145 may be accessible to a healthcare provider of a member (also referred to herein as a patient), and in some cases, include member information associated with the healthcare provider that provided a treatment to the member. In some aspects, the provider database 145 may be accessible to an insurance provider associated with the member. The member database 150 may correspond to any type of known database, and the fields of the electronic records may be formatted according to the type of database used to implement the member database 150. Non-limiting examples of the types of database architectures that may be used for the member database 150 include a relational database, a centralized database, a distributed database, an operational database, a hierarchical database, a network database, an object-oriented database, a graph database, a NoSQL (non-relational) database, etc. In some cases, the member database 150 may include an entire healthcare history or journey of a member, whereas the provider database 145 may provide a snapshot of a member's healthcare history with respect to a healthcare provider. In some examples, the electronic records stored in the member database 150 may correspond to a collection or aggregation of electronic records from any combination of provider databases 145 and entities involved in the member's healthcare delivery (e.g., a pharmaceutical distributor, a pharmaceutical manufacturer, etc.).

In some aspects, variables associated with the gaps-in-care described herein (e.g., stored in the provider database 145 and/or the member database 150) may include insurance claims, pharmacy records, laboratory results, and utilization history.

The provider database 145 and/or the member database 150 may include chronic disease indicators recorded for each member using a database format associated with the provider database 145 and/or the member database 150. In some aspects, the provider database 145 and/or the member database 150 may support diagnosis and procedure codes classified according to the International Classification of Diseases 10th revision (ICD-10) and Current Procedure Terminology 4th revision (CPT-4) codes. In some aspects, the provider database 145 and/or the member database 150 may support the use of Generic Product Identifier (GPI) and National Drug Code (NDC) Directory information for common diabetes medications. The provider database 145 and/or member database 150 may include demographic information, including age, gender, race, and geography, identified using claims data. The provider database 145 and/or member database 150 may include data such as proportion of days covered (PDC), calculated as a ratio of the number of days in a period covered to the number of days in a given period for each member and corresponding medication.

Aspects of the present disclosure may support new, engineered features of gaps in diabetes care not addressed by the above variables. For example, the server 135 (e.g., using the care gap management engine 182 described later herein) may support feature engineering of care gaps spanned the following example disease management domains: medication regimen optimization, prescription adherence, preventative screenings and lifestyle management, and, for diabetes, blood glucose monitoring. It should be noted that other medical conditions could be managed by tracking other related or applicable biomarkers. In the example of diabetes, the server 135 may engineer example specialized gaps in care described herein using a combination of ICD-10, CPT4, GPI, and NDC codes, healthcare utilization history, and adherence to a prescribed management device including self-monitoring of blood glucose (SMBG), a connected glucose meter (CGM), or diabetes test strips. Aspects of the present disclosure support classification of care gap status according to the following examples: [1] eligible open (‘care gap open’), [2] eligible closed (‘care gap closed’), and [3] ineligible.

Various example aspects of the provider database 145 and/or the member database 150 with respect to the present disclosure are described in Exhibit A, for example, at Section II, “Database Description” and Supplementary Expanded Methods, Section II, “Database Description”. Various example aspects of gaps-in-care are described in Exhibit A, for example, at Supplementary Table 1, “Diabetes Gaps in Care”.

The server 135 may include a processor 160, a network interface 165, a database interface 170, and a memory 175. In some examples, components of the server 135 (e.g., processor 160, a network interface 165, a database interface 170, and a memory 175) may communicate via a system bus (e.g., any combination of control busses, address busses, and data busses) included in the server 135. Aspects of the processor 160, network interface 165, database interface 170, and memory 175 may support example functions of the server 135 as described herein. For example, the server 135 may transmit packets to (or receive packets from) one or more other devices (e.g., one or more communication devices 105, another server 135, the provider database 145, the provider database 150) via the communication network 140. In some aspects, via the network interface 165, the server 135 may transmit database queries to one or more databases (e.g., provider database 145, member database 150) of the system 100, receive responses associated with the database queries, or access data associated with the database queries.

In some aspects, via the network interface 165, the server 135 may transmit one or more reports 155 described herein to one or more communication devices 105 of the system 100. The network interface 165 may include, for example, any combination of network interface cards (NICs), network ports, associated drivers, or the like. Communications between components (e.g., processor 160, network interface 165, database interface 170, and memory 175) of the server 135 and other devices (e.g., one or more communication devices 105, the provider database 145, the provider database 150, another server 135) connected to the communication network 140 may, for example, flow through the network interface 165.

The processors described herein (e.g., processor 110 of the communication device 105, processor 160 of the server 135) may correspond to one or many computer processing devices. For example, the processors may include a silicon chip, such as a Field Programmable Gate Array (FPGA), an Application-Specific Integrated Circuit (ASIC), any other type of Integrated Circuit (IC) chip, a collection of IC chips, or the like. In some aspects, the processors may include a microprocessor, a Central Processing Unit (CPU), a Graphics Processing Unit (GPU), or plurality of microprocessors configured to execute the instructions sets stored in a corresponding memory (e.g., memory 120 of the communication device 105, memory 175 of the server 135). For example, upon executing the instruction sets stored in memory 120, the processor 110 may enable or perform one or more functions of the communication device 105. In another example, upon executing the instruction sets stored in memory 175, the processor 160 may enable or perform one or more functions of the server 135.

The processors described herein (e.g., processor 110 of the communication device 105, processor 160 of the server 135) may utilize data stored in a corresponding memory (e.g., memory 120 of the communication device 105, memory 175 of the server 135) as a neural network. The neural network may include a machine learning architecture. In some aspects, the neural network may be or include one or more classifiers. In some other aspects, the neural network may be or include any machine learning network such as, for example, a deep learning network, a convolutional neural network, or the like. Some elements stored in memory 120 may be described as or referred to as instructions or instruction sets, and some functions of the communication device 105 may be implemented using machine learning techniques. In another example, some elements stored in memory 175 may be described as or referred to as instructions or instruction sets, and some functions of the server 135 may be implemented using machine learning techniques.

In some aspects, the processors (e.g., processor 110, processor 160) may support machine learning model(s) 184 which may be trained and/or updated based on data (e.g., training data 186) provided or accessed by any of the communication device 105, the server 135, the provider database 145, and the member database. The machine learning model(s) 184 may be built and updated by the care gap management engine 182 based on the training data 186 (also referred to herein as training data and feedback). For example, the machine learning model(s) 184 may be trained with feature vectors of members (e.g., accessed from provider database 145 or member database 150) for which adherence to one or more actions reduced a corresponding gap-in-care and/or achieved one or more impacts (e.g., cost impact, clinical impact, etc.).

The machine learning model(s) 184 may be provided in any number of formats or forms. Example aspects of the machine learning model(s) 184, such as generating (e.g., building, training) and applying the machine learning model(s) 184, are described with reference to the figure descriptions herein.

Non-limiting examples of the machine learning model(s) 184 include Decision Trees, gradient-boosted decision tree approaches (GBMs), Support Vector Machines (SVMs), Nearest Neighbor, and/or Bayesian classifiers, and neural-network-based approaches.

In an example, the machine learning model(s) 184 may include ensemble classification models (also referred to herein as ensemble methods) such as gradient boosting machines (GBMs). Gradient boosting techniques may include, for example, the generation of decision trees one at a time within a model, where each new tree may support the correction of errors generated by a previously trained decision tree (e.g., forward learning). Gradient boosting techniques may support, for example, the construction of ranking models for information retrieval systems. A GBM may include decision tree-based ensemble algorithms that support building and optimizing models in a stage-wise manner.

According to example aspects of the present disclosure described herein, the machine learning model(s) 184 may include Gradient Boosting Decision Trees (GBDTs). Gradient boosting is a supervised learning technique that harnesses additive training and tree boosting to correct errors made by previous models, or regression trees.

The machine learning model(s) 184 may include extreme gradient boosting (CatBoost) models. CatBoost is an ensemble learning method based on GBDTs. In some cases, CatBoost methods may have improved performance compared to comparable random forest-based methods. CatBoost methods are easily tunable and scalable, offer a higher computational speed in comparison to other methods, and are designed to be highly integrable with other approaches including Shapley Additive Explanations (SHAP) values.

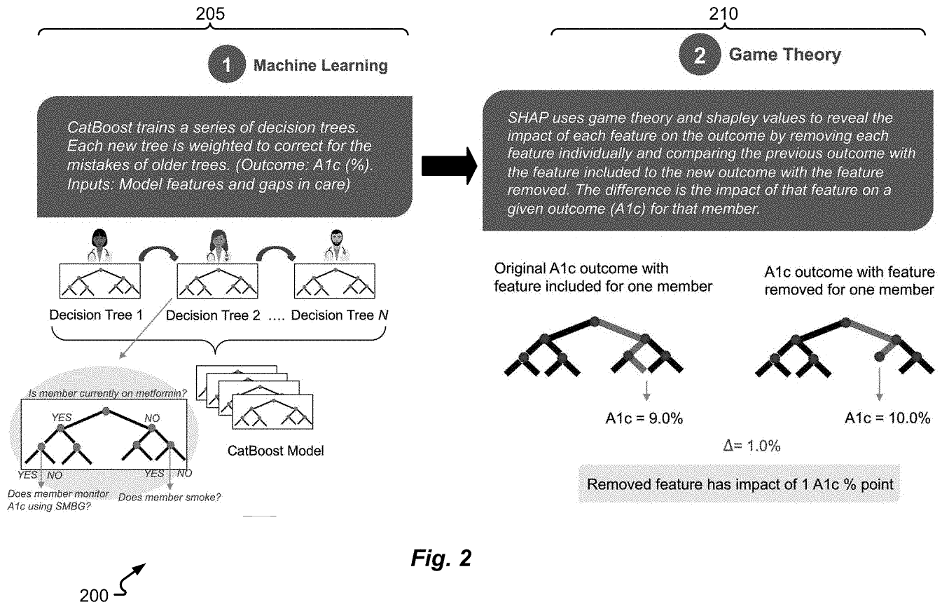

Examples implementations of the GBDTs and CatBoost models are described herein with reference to FIG. 2.

In some aspects, the machine learning model(s) 184 may include ensemble classification models (also referred to herein as ensemble methods) such as random forests. Random forest techniques may include independent training of each decision tree within a model, using a random sample of data. Random forest techniques may support, for example, medical diagnosis techniques described herein using weighting techniques with respect to different data sources.

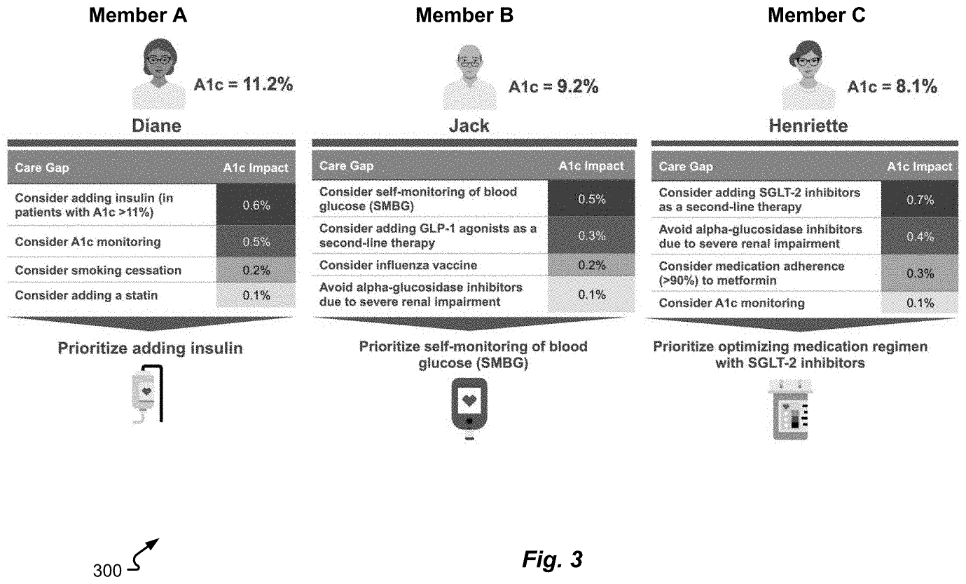

Various example aspects of the machine learning model(s) 184, inputs to the machine learning model(s) 184, and the training data 186 with respect to the present disclosure are described in Exhibit A, for example, at FIG. 3, “SHAPLEY Values Drive Member-Level Intervention Pairing”, Section III, “Machine Learning and Game Theory: A1c Prediction”, Supplementary Expanded Methods, Section III, “Machine Learning and Game Theory: A1c Prediction”, Supplementary Expanded Methods, Section IV, “Clinical Interventions”, and Supplementary FIG. 1, “Consort diagram of the study population.”

The memory described herein (e.g., memory 120, memory 175) may include any type of computer memory device or collection of computer memory devices. For example, a memory (e.g., memory 120, memory 175) may include a Random Access Memory (RAM), a Read Only a Memory (ROM), a flash memory, an Electronically-Erasable Programmable ROM (EEPROM), Dynamic RAM (DRAM), or any combination thereof

The memory described herein (e.g., memory 120, memory 175) may be configured to store instruction sets, neural networks, and other data structures (e.g., depicted herein) in addition to temporarily storing data for a respective processor (e.g., processor 110, processor 160) to execute various types of routines or functions. For example, the memory 175 may be configured to store program instructions (instruction sets) that are executable by the processor 160 and provide functionality of any of the engines (e.g., feature embedding engine 179, member grouping engine 180, care gap management engine 182, reporting engine 188, etc.) described herein.

The memory described herein (e.g., memory 120, memory 175) may also be configured to store data or information that is useable or capable of being called by the instructions stored in memory. Examples of data that may be stored in memory 175 for use by components thereof include machine learning model(s) 184 and/or training data 186 described herein.

Any of the engines (e.g., feature embedding engine 179, member grouping engine 180, care gap management engine 182, reporting engine 188, etc.) described herein may include a single or multiple engines.

With reference to the server 135, the memory 175 may be configured to store instruction sets, neural networks, and other data structures (e.g., depicted herein) in addition to temporarily storing data for the processor 160 to execute various types of routines or functions. The illustrative data or instruction sets that may be stored in memory 175 may include, for example, database interface instructions 176, an electronic record filter 178 (also referred to herein as a feature vector filter), a feature embedding engine 179, a care gap management engine 182, and a reporting engine 188. In some examples, the reporting engine 188 may include data obfuscation capabilities 190 via which the reporting engine 188 may obfuscate, remove, redact, or otherwise hide personally identifiable information (PII) from a report 155 prior to transmitting the report 155 to another device (e.g., communication device 105).

In some examples, the database interface instructions 176, when executed by the processor 160, may enable the server 135 to send data to and receive data from the provider database 145, the member database 150, or both. For example, the database interface instructions 176, when executed by the processor 160, may enable the server 135 to generate database queries, provide one or more interfaces for system administrators to define database queries, transmit database queries to one or more databases (e.g., provider database 145, the member database 150), receive responses to database queries, access data associated with the database queries, and format responses received from the databases for processing by other components of the server 135.

The server 135 may use the electronic record filter 178 in connection with processing data received from the various databases (e.g., provider database 145, member database 150). For example, the electronic record filter 178 may be leveraged by the database interface instructions 176 to filter or reduce the number of electronic records (e.g., feature vectors) provided to any of the feature embedding engine 179 or the care gap management engine 182. In an example, the database interface instructions 176 may receive a response to a database query that includes a set of feature vectors (e.g., a plurality of feature vectors associated with different members). In some aspects, any of the database interface instructions 176, the feature embedding engine 179, or the care gap management engine 182 may be configured to utilize the electronic record filter 178 to reduce (or filter) the number of feature vectors received in response to the database query, for example, prior to processing data included in the feature vectors.

The feature embedding engine 179 may receive, as input, sequences of medical terms extracted from claim data (e.g., medical claims, pharmacy claims) for each member. In an example, the feature embedding engine 179 may process the input using neural word embedding algorithms such as Word2vec. In some examples, the feature embedding engine 179 may process the input using Transformer algorithms (e.g., algorithms associated with language models such as Bidirectional Encoder Representations from Transformers (BERT) or Generative Pre-trained Transformer (GPT) or graph convolutional transformer (GCT)) and respective attentional mechanisms. In some aspects, based on the processing, the feature embedding engine 179 may compute and output respective dimension weights for the medical terms. In some aspects, the dimension weights may include indications of the magnitude and direction of the association between a medical code and a dimension. In an example, the feature embedding engine 179 may compute an algebraic average of all the medical terms for each member over any combination of dimensions (e.g., over all dimensions). In some aspects, the algebraic average may be provided by the feature embedding engine 179 as additional feature vectors in a predictive model described herein (e.g., classifier).

The member grouping engine 180, when executed by the processor 160, may enable the server 135 to group data records of various members according to a common value(s) in one or more fields of such data records. For example, the member grouping engine 180 may group electronic records based on commonalities in parameters such as health conditions (e.g., diagnosis of diabetes, open gaps-in-care, closed gaps-in-care, suggested actions associated with closing a gap-in-care, impact associated with at least partially closing the gap-in-care, etc.), medical treatment histories, prescriptions, healthcare providers, locations (e.g., state, city, ZIP code, etc.), gender, age range, medical claims, pharmacy claims, lab results, medication adherence, demographic data, social determinants (also referred to herein as social indices), biomarkers, behavior data, engagement data, historical gap-in-care data, machine learning model-derived outputs, combinations thereof, and the like.

The reporting engine 188, when executed by the processor 160, may enable the server 135 to output one or more reports 155 based on data generated by any of the feature embedding engine 179, the member grouping engine 180, or the care gap management engine 182. The reporting engine 188 may be configured to generate reports 155 in various electronic formats, printed formats, or combinations thereof. Some example formats of the reports 155 may include HyperText Markup Language (HTML), electronic messages (e.g., email), documents for attachment to an electronic message, text messages (e.g., SMS, instant messaging, etc.), combinations thereof, or any other known electronic file format. Some other examples include sending, for example, via direct mail, a physical representation (e.g., a letter) of the report 155.

The reporting engine 188 may also be configured to hide, obfuscate, redact, or remove PII data from a report 155 prior to transmitting the report 155 to another device (e.g., a communication device 105). In some aspects, a communication device 105 may also be configured to hide, obfuscate, redact, or remove PII data from direct mail (e.g., a letter) prior to generating a physical representation (e.g., a printout) of a report 155. In some examples, the data obfuscation may include aggregating electronic records to form aggregated member data that does not include any PII for a particular member or group of members. In some aspects, the aggregated member data generated by the data obfuscation may include summaries of data records for member groups, statistics for member groups, or the like.

Example illustrative aspects of the system 100 are described with reference to the following figures described herein.

FIG. 2 illustrates an example of machine learning and game theory based approaches in accordance with aspects of the present disclosure.

Referring the FIG. 2, aspects of the present disclosure include a machine learning based approach 205 and a game theory based approach 210. In some aspects, the machine learning based approach 205 and the game theory based approach 210 may be implemented by aspects of the server 135, the machine learning model(s) 184, and the care gap management engine 182 described with reference to FIG. 1.

The machine learning based approach 205 includes prioritizing gaps in care on HbA1c by training a series of decision trees (e.g., decision tree 1 through decision tree N) on clinically-relevant features that may influence diabetes status. As illustrated in FIG. 2, an example of decision tree 2 includes factors associated with whether a member is currently on metformin, whether the member monitors A1c using SMBG, and whether the member smokes.

In some aspects, the machine learning based approach 205 incorporates an A1c prediction model generated using a CatBoost model. In some aspects, the machine learning based approach 205 includes using a CatBoost model to predict A1c for each member in a member population.

The machine learning based approach 205 includes tuning all model features (e.g., of the CatBoost model) through hyperparameter optimization.

According to example aspects of the present disclosure, the boosting method of the machine learning based approach 205 is analogous to a team of physicians leveraging a member's history and clinical findings to increase or decrease the pre-test probability of a diagnosis. Other aspects of the machine learning based approach 205 may be analogous to a physician arriving at a prognosis versus a team of physicians combining individual knowledge to arrive at the most clinically-accurate decision.

Aspects of the present disclosure support applying the game theory based approach 210 to the described gradient boosting model (e.g., CatBoost model) to generate member-level predictions. For example, using SHAP, the server 135 may assign each model feature an importance value for a given prediction. In an example, using the SHAP values (also referred to herein as Shapely values), the server 135 may generate an understanding of the degree to which each actor (e.g., each model feature) fairly contributes to a shared mission (e.g., A1c).

SHAP values may attribute an importance value by comparing what a model predicts with and without a feature present, maintaining consistency and accuracy. As the order in which a model sees each feature can affect prediction of the outcome, a significant combinatorial problem occurs. SHAP values use partial differential calculus and approximations to speed up the process of attributing importance values, generating a model output agnostic of feature importance.

Aspects of the game theory based approach 210 include applying SHAP values as a proxy for the recoverable A1c in a member population by approximating the degree to which closing a particular gap in care yields a treatment benefit. Accordingly, for example, the game theory based approach 210 may support predicting the impact of a given feature (e.g., whether the member is on metformin, whether the member monitors A1c using SMBG, whether the member smokes, etc.) on a member's predicted A1c.

For example, the game theory based approach 210 includes calculating SHAP values for members in an analyzed population with a given care gap open versus closed. In an example, the game theory based approach 210 includes using the delta between the different SHAP values (e.g., care gap open vs. care gap closed) to assess the clinical impact of closing each gap in care for a member with Type 2 diabetes. In some aspects, the game theory based approach 210 provides improved accuracy in estimating the heterogeneous treatment effects of closing A1c. For example, as illustrated in the example of FIG. 2, removing a feature (e.g., whether the member is on metformin, whether the member monitors A1c using SMBG, whether the member smokes, etc.) included for a member may have an impact of one (1) A1c % point.

Various example aspects of the machine learning based approach 205 and game theory based approach 210 with respect to the present disclosure are described in Exhibit A, for example, at FIG. 1 (page 9), “Machine learning and game theory leveraging CatBoost and SHAP enable member-level predictions”, Section III, “Machine Learning and Game Theory: A1c Prediction”, Supplementary Expanded Methods, Section III, “Machine Learning and Game Theory: A1c Prediction”.

FIG. 3 illustrates an example of actions a member may take in association with closing a gap-in-care in accordance with aspects of the present disclosure.

In some examples, the actions may be calculated and predicted by aspects of a communication device 105, a server 135, a care gap management engine 182, and a machine learning model(s) 184 described with reference to FIG. 1. In some aspects, the actions illustrated may be referred to as ‘next best actions’ for a member (e.g., member A, member B, etc.) to take in connection with at least partially closing a current gap-in-care for the member. In some example aspects, the server 135 may generate and order (and a communication device 105 may display) the actions in an order corresponding to A1c impact. In some examples, the server 135 may provide a report 155 inclusive of the actions and corresponding A1c impacts to a communication device 105 (e.g., of a member, a healthcare provider, an insurance provider, etc.), a provider database 145, and/or a member database 150. In some cases, calculation and prediction of the actions may be based on correlations between closing gaps-in-care and A1c impact, which may be individualized for each member or a particular group of members.

In an example, current gaps-in-care for member A and A1c impacts associated with taking an action that addresses a corresponding gap-in-care may include adding insulin (having an A1c impact of 0.6%), A1c monitoring (having an A1c impact of 0.5%), smoking cessation (having an A1c impact of 0.2%), and adding a statin (having an A1c impact of 0.1%). In some aspects, the server 135 (e.g., care gap management engine 182) may generate an output indicating a recommendation for member A to prioritize adding insulin.

In another example, current gaps-in-care for member B and A1c impacts associated with taking an action that addresses the corresponding gap-in-care may include SMBG (having an A1c impact of 0.5%), adding GLP-1 agonists as a second-line therapy (having an A1c impact of 0.3%), receiving the influenza vaccine (having an A1c impact of 0.2%), and avoiding alpha-glucosidase inhibitors (having an A1c impact of 0.1%). In some aspects, the server 135 (e.g., care gap management engine 182) may generate an output indicating a recommendation for member B to prioritize SMBG.

In another example, current gaps-in-care for member C and A1c impacts associated with taking an action that addresses a corresponding gap-in-care may include adding SGLT-2 inhibitors as a second-line therapy (having an A1c impact of 0.7%), avoiding alpha-glucosidase inhibitors (having an A1c impact of 0.4%), achieving medication adherence of greater than 90% (having an A1c impact of 0.3%), and A1c monitoring (having an A1c impact of 0.1%). In some aspects, the server 135 (e.g., care gap management engine 182) may generate an output indicating a recommendation for member C to prioritize optimize medication regimen with SGLT-2 inhibitors.

Various example aspects of the training data 186 with respect to the present disclosure are described in Exhibit A, for example, at FIG. 1 (page 9), “Next Best Actions are unique for each member”, FIG. 3 (page 14), “SHAPLEY Values Drive Member-Level Intervention Pairing”, Section III, “Machine Learning and Game Theory: A1c Prediction”, and Supplementary Expanded Methods, Section III, “Clinical Interventions”.

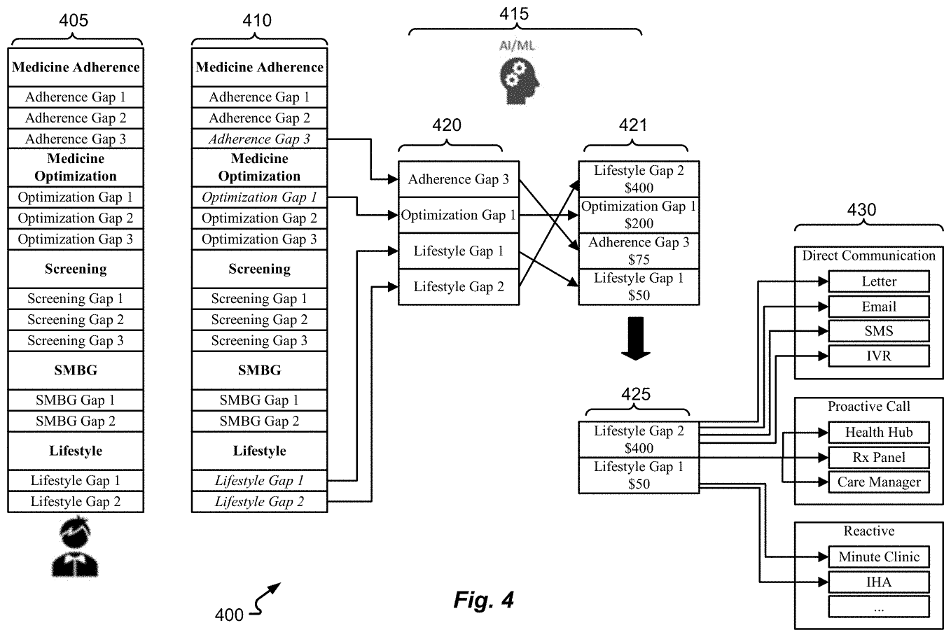

FIG. 4 illustrates an example block diagram 400 that supports gap-in-care valuation, prioritization, and channel selection in accordance with aspects of the present disclosure.

In some examples, the block diagram 400 may be implemented by aspects of a communication device 105, a server 135, and/or a care gap management engine 182 described with reference to FIG. 1. According to example aspects of the present disclosure, gaps in diabetes care are unique for each member and variably influence HbA1c.

The server 135 (e.g., care gap management engine 182) may identify, from candidate gaps-in-care 405, open gaps-in-care (italicized in 410) and closed gaps-in-care (non-italicized in 410) associated with a member (e.g., for each member). For example, the server 135 may identify the open gaps-in-care and closed gaps-in-care from electronic records stored in the provider database 145 and/or member database 150. In an example, the server 135 may identify, for a member (e.g., a diabetic), which of the candidate gaps-in-care 405 are open or closed. In some examples, the candidate gaps-in-care 405 may include greater than seventy (70) gaps-in-care.

The server 135 may compute values for a group 420 of open gaps-in-care, for example, using a machine learning network 415. The machine learning network 415 may include examples of aspects of the machine learning model(s) 184 and care gap management engine 182. In an example, the values may include corresponding clinical impacts (e.g., A1c impact) and/or cost impacts (e.g., financial amounts) that may be obtained for closing each open gap-in-care of the group 420. The server 135 may provide the values and corresponding open gaps-in-care in a group 421. In an example, the server 135 may order (e.g., ascending order, descending order) the open gaps-in-care according to the respective values, as illustrated at group 421.

The server 135 may identify, from among the open gaps-in-care included in group 421, gaps-in-care that are clinically relevant to one another. For example, the server 135 may identify that, from among group 421, “Lifestyle Gap 2” and “Lifestyle Gap 1” are clinically relevant to one another. The server 135 may further assign “Lifestyle Gap 2” and “Lifestyle Gap 1” to a group 425.

The server 135 may identify communication channels 430 for providing a communication to the member. In an example, the communication may describe an action(s) associated with at least partially closing one or more current gaps-in-care (e.g., “Lifestyle Gap 2”, “Lifestyle Gap 1”, etc.) of the group 425. In some examples, the communication may describe a corresponding impact (e.g., cost impact, clinical impact) associated with at least partially closing the one or more current gaps-in-care. In an example of identifying communication channels 430, the server 135 may increase the probabilities of successful outcomes through selective channel assignment that incorporates member preferences.

The communication channels 430 may include aspects of the example communication channels described herein (e.g., email, direct mail, SMS, an automated outbound calling campaign, a personalized phone call from a provider and/or care manager of the member, etc.). In some aspects, the selected communication channel and/or the timing of a communication is based on a location of the member. For example, if a member has an open care gap related to medication adherence or filling of a prescription, and a member is detected as being near a pharmacy location where the medication is available for pick-up, the server 135 may provide a message or communication to a communication device 105 of the member prompting the member to pick up the medication. In some aspects, the server 135 may provide the communication to a communication device 105 of the member. In some aspects, the server 135 may provide the same communication (or a portion thereof) to a communication device 105 of a healthcare provider of the member and/or an insurance provider, the provider database 145, and/or the member database 150.

In some aspects, the server 135 (e.g., care gap management engine 182) may leverage machine learning algorithms for providing communications to the member. For example, aspects of the present disclosure may support achieving consistent and effective connections (e.g., communications) and feedback signals (e.g., an identification of communication channels that are relatively effective and/or ineffective) with respect to providing communications to a member.

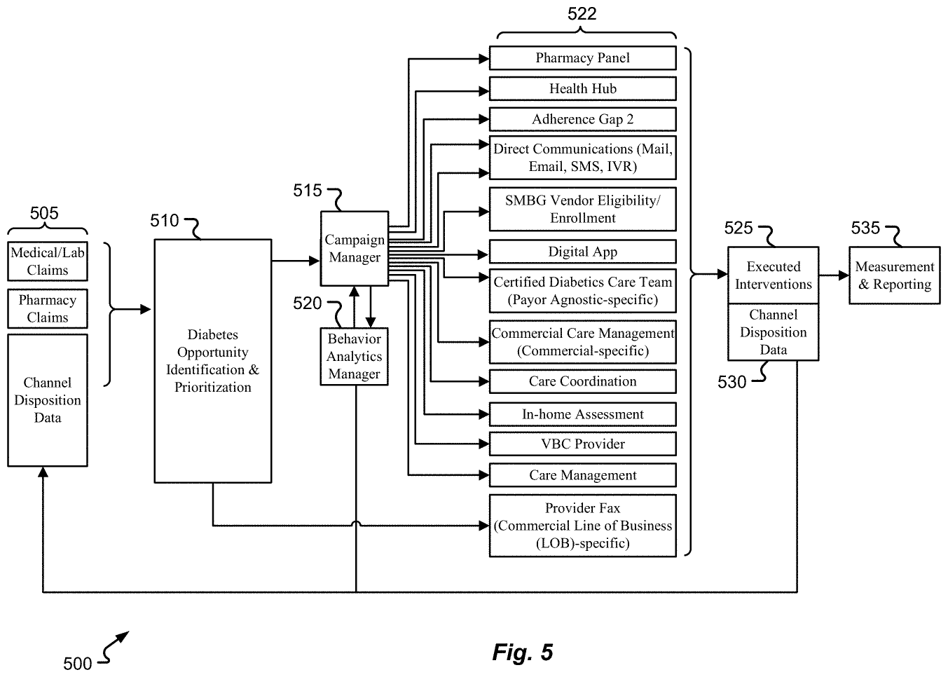

FIG. 5 illustrates an example operational flow 500 that supports tracking chronic conditions in accordance with aspects of the present disclosure.

In some examples, the operational flow 500 may be implemented by aspects of a communication device 105, a server 135, and/or a care gap management engine 182 described with reference to FIG. 1. In some aspects, the operational flow 500 may be implemented by an operational model, which may be implemented by aspects of a machine learning model(s) 184 described with reference to FIG. 1. In some aspects, portions of the operational flow 500 may be implemented by a server 135.

In some other aspects, the operational flow 500 may be implemented by and distributed over multiple servers 135. For example, a server 135 may implement data inputs and aggregation (as will be described with reference to 505), model running at scale (as will be described with reference to 510), intervention routing (as will be described with reference to 515 and 520), etc.

At 505, the server 135 may retrieve and aggregate data associated with a set of members. In an example, the set of members may include members diagnosed with a target condition (e.g., Type 1 diabetes, Type 2 diabetes, etc.). In some aspects, the set of members may include members susceptible to being diagnosed with the target condition (e.g., at risk of the target condition within a temporal period).

For example, the server 135 may retrieve electronic records of members from the provider database 145 and/or member database 150 described with reference to FIG. 1. In some aspects, the electronic record may include raw and unorganized information. In an example, the server 135 may retrieve the data and/or the data may be pushed through a data pipeline to the server 135 continuously, based on a schedule (e.g., monthly, weekly, daily, etc.), and/or based on a trigger condition.

The data may include medical/lab claims data (e.g., medical and prescription data), pharmacy claims data, and channel disposition data. In some aspects, the channel disposition data may support features in which the server 135 (e.g., care gap management engine 182) may manage and close gaps-in-care as described herein. In some aspects, the channel disposition data may trigger member care ‘journeys’ as described herein.

In an example, the medical/lab claims data may be organized in a table form in a database (e.g., provider database 145, member database 150, etc.). In some examples, the server 135 may derive a table (e.g., a member health state table) from the medical/lab claims data and/or the pharmacy data, based on which the server 135 may assess gaps-in-care associated with a member(s). The gaps-in-care may be associated with one of three states: ineligible (e.g., the care gap does not apply to the member), closed (e.g., the care gap does apply to the member, the member has already met the requirements to address the care gap), and open (e.g., the care gap does apply to this member, the member has not yet met the requirements to address the care gap).

The channel disposition data may include historical data (e.g., previous information) on member responses to past campaigns provided by a campaign manager 515 (e.g., personnel, a communication device 105 of a user associated with a medical provider or insurance provider, etc.). The campaign manager 515 may be implemented by, for example, aspects of the server 135, the care gap management engine 182, and a communication device 105. The server 135 may review, for each previous communication to a member (e.g., with respect to a treatment plan, a diagnosis, a gap-in-care, etc.), member dispositions associated with a corresponding response by the member. For example, the server 135 may review the channel disposition data to identify any gaps-in-care for which a communication has already been provided to a member. In some aspects, the channel disposition data may include a logical hierarchy for use in closing gaps-in-care.

The server 135 may unify the medical/lab claims data, pharmacy claims data, and channel disposition data into a single source of information (e.g., a single database, a single table, etc.). In some aspects, the single source of information may be organized according to a unique identifier associated with each member. The single source of information may be referred to as a unify table. In some aspects, the unique identifier may be different from a member ID, as in some cases, a member ID may change over time.

At 510, the server 135 may process the unify table. For example, the server 135 may organize and format information in the unify table. In some aspects, the server 135 may generate a machine learning model based on the information in the unify table. In some aspects, the server 135 may apply aspects of the machine learning based approach 205 and game theory based approach 210 described herein to generate the machine learning model.

For example, at 510, the server 135 may deploy CatBoost as described herein. In some aspects, at 515, the server 135 may measure the health of members with diabetes based on a biomarker such as HbA1c.

In some aspects, at 510, the server 135 may deploy SHAP, aspects of which are described herein. For example, using SHAP, the server 135 may analyze the correlative impact of features (e.g., predictive of HbA1c) associated with a member on the outcome of A1c. By deploying SHAP, the server 135 may determine, for each member and each feature, a correlative impact of all of the features with respect to an outcome (e.g., a clinical impact, a cost impact, etc.). For example, the server 135 may estimate how much closing one or more open gaps-in-care may reduce HbA1C and create medical cost savings through reducing A1c.

According to example aspects of the present disclosure, the model (e.g., a machine learning model(s) 184) implemented at 510 may be a pretrained machine learning model. For example, the pretrained machine learning model may have been trained from data associated with a previous run of the operational flow 500. In some examples, the model implemented at 510 may be prioritized based on clinical values, care considerations, and defined care gaps.

In some aspects, at 510, the server 135 may organize the generated data (e.g., following SHAP deployment) for processing at the campaign manager 515. Aspects of campaign manager 515 may be referred to as intervention routing.

The server 135 may organize the data based on ranking information (e.g., a value generated by CatBoost and/or SHAP models described herein) indicative of a probability of closing a gap-in-care corresponding to a member. In an example, at campaign manager 515, the server 135 may determine or calculate a level of outreach (e.g., High, Medium, Low) for providing a communication to a member. In some aspects, the server 135 may allocate a relatively higher level of outreach for members based on value (e.g., cost impact, clinical impact) associated with addressing a gap-in-care and/or type of gap-in-care. In an example, the server 135 may allocate a relatively higher level of outreach for a gap-in-care based on a comparison of the value (e.g., cost impact, clinical impact) associated with reducing and/or eliminating the gap-in-care to the probability of success of that outreach. For example, the server 135 may allocate a relatively higher level of outreach for a gap-in-care when the value (e.g., cost impact, clinical impact) associated with reducing and/or eliminating the gap-in-care is greater than or equal to the cost (e.g., financial cost) of the outreach multiplied by the probability of success of that outreach.

In some aspects, at campaign manager 515 and/or behavior analytics manager 520, the server 135 may review, assess, and/or determine communication parameters (e.g., a communication frequency, a communication channel, etc.) associated with providing communications to the member. For example, the server 135 may review and/or assess handling past member dispositions associated with previous communications to the member.

For example, at campaign manager 515 and behavior analytics manager 520 (e.g., personnel, a communication device 105 of a user associated with a medical provider or insurance provider, etc.), the server 135 may identify characteristics associated with previous communications (e.g., quantity, frequency, member responses, etc.). Accordingly, for example, the server 135 may identify a communication channel (e.g., email, text, voice communication, etc.) and communication frequency to determine and implement a communication approach which may provide member convenience. In some aspects, the communication approach may support an improved member adherence to actions for closing gaps-in-care associated with the member.

At 522, the server 135 may determine intervention delivery to a member. For example, at 520, the server 135 may identify a communication channel for communicating with a member, as determined at campaign manager 515. In some aspects, at 522, the server 135 may transmit instructions to delivery channel systems (e.g., a pharmacy, a health hub, an application, a direct mail service, an email server, etc.) to provide a communication to the member, in which the communication includes actions associated with reducing a gap-in-care.

At 525, 530, and 535, the server 135 may handle interventions implemented at 520. For example, at 525, the server 135 may execute the interventions determined at campaign manager 515 (e.g., provide communications with a member(s) based on determined communications channels, communication frequencies, etc. described herein). Handling the interventions may be specific to communications channel and/or intervention. In some cases, at 525, the server 135 may track and/or record outcomes (e.g., health improvements, closures of gaps-in-care, etc.) and member dispositions (e.g., member responses) related to executed interventions. At 530, the server 135 may identify channel disposition data (e.g., dispositions associated with previous communications/outreach to a member(s)) and provide the disposition data to the campaign manager 515 and/or behavior analytics manager 520 (e.g., creating a feedback loop).

At 535, the server 135 may maintain a record of communications provided to members and/or dispositions corresponding to the provided communications. In some aspects, the server 135 may store the record in the provider database 145 and/or member database 150. In some aspects, at 535, the server 135 may provide measurement and reporting records. For example, the server 135 may provide operational reporting including: outcomes, outreach methods, and care gap categories associated with executed interventions.

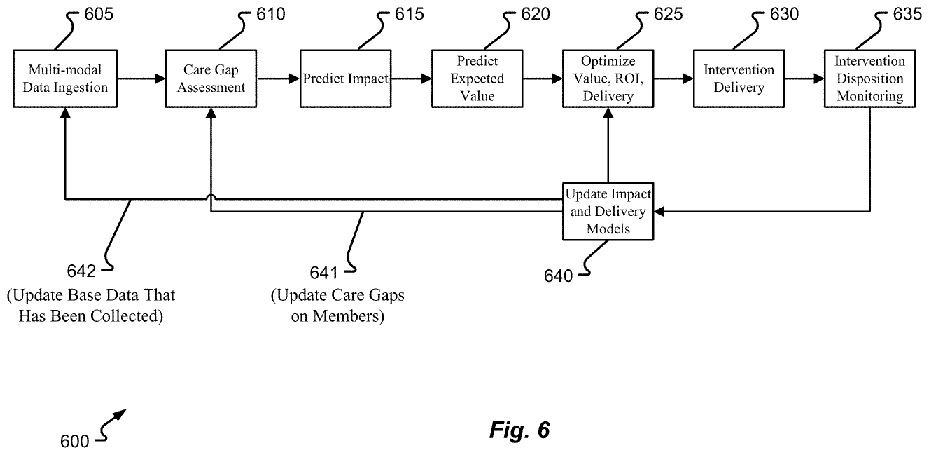

FIG. 6 illustrates an example of a process flow 600 that supports tracking chronic conditions in accordance with aspects of the present disclosure. For example, process flow 600 may support observing member behavior and managing a care gap associated with the member.

In some examples, process flow 600 may be implemented by aspects of server 135 or a care gap management engine 182 described with reference to FIG. 1. For example, process flow 600 may implement aspects of machine learning based approach 205 and/or game theory based approach 210 described with reference to FIG. 2. In some aspects, process flow 600 may implement aspects described with reference to any figure described herein.

In some aspects, the process flow 600 may be implemented as a single model (e.g., a single machine learning model 184) or a combination of models (e.g., multiple machine learning models 184). In some aspects, process flow 600 may support multiple models capable of training one another (e.g., a recursive learning network). In some aspects, one or more models may be implemented algorithmically or as a machine learning model described herein.

In the following description of the process flow 600, the operations may be performed in a different order than the order shown, or the operations may be performed in different orders or at different times. Certain operations may also be left out of the process flow 600, or other operations may be added to the process flow 600.

It is to be understood that while a server 135 is described as performing a number of the operations of process flow 600, any device (e.g., another server 135, a combination of servers 135, a communication device 105, a combination of a server 135 and a communication device 105) may perform the operations shown.

At 605, the server 135 may implement multi-modal data ingestion. For example, at 605, the server 135 may retrieve and aggregate data associated with a set of members (e.g., as described with reference to 505 of FIG. 5).

At 610, the server 135 may assess gaps-in-care (e.g., current gaps-in-care, new gaps-in-care, etc.) associated with a member(s). For example, the gaps-in-care may be defined as a difference between a current health-related behavior of the member and a guideline behavior for the member.

In an example, at 610, the server 135 may identify one or more actions for the member that, if followed, are capable of at least partially closing the current gap-in-care within a clinically-defined period of time for the member.

At 615, the server 135 may determine or predict an impact (e.g., a clinical impact, a cost impact, etc. described herein) associated with at least partially closing the gaps-in-care for the member according to the one or more actions. For example, the server 135 may implement aspects described with reference to 510 of FIG. 5.

At 620, the server 135 may predict an expected value (e.g., a cumulative A1c value) associated with at least partially closing the gaps-in-care for the member according to the one or more actions. For example, the server 135 may implement aspects described with reference to 510 of FIG. 5.

At 625, the server 135 may optimize value (e.g., clinical impact, cost impact, etc.), ROI (e.g., for an insurance provider, a medical provider, a member, etc.), and delivery (e.g., communications via a channel) associated with at least partially closing the gaps-in-care. For example, the server 135 may implement aspects described with reference to 510 through 520 of FIG. 5.

At 630, the server 135 may implement intervention delivery. For example, at 630, the server 135 may implement aspects described with reference to intervention delivery at 520 of FIG. 5.

At 635, the server 135 may monitor intervention dispositions associated with delivered interventions. For example, at 630, the server 135 may implement aspects described with reference to intervention handling at 525 through 535 of FIG. 5.

At 640, the server 135 may update impact and delivery models. For example, at 640, the server 135 may implement aspects described with reference to channel disposition data at 505 of FIG. 5.

In an example, the server 135 may output updated gaps-in-care associated with one or more members. For example, the server 135 may output an indication 641 of progress completion (e.g., uncompleted, completed, a completion percentage) associated with gaps-in-care associated with one or more members.

In another example, at 640, the server 135 may output updated baseline data (e.g., baseline biomarkers) for ingestion at 605. In some aspects, the updated baseline data (e.g., at 642) may be associated with health improvements (e.g., improved baseline HbA1c levels, A1c levels, etc.) corresponding to a member.

### Program Mechanics Overview

Program mechanics associated with the examples of observing member behavior and managing a care gap as described herein are provided. The program mechanics may be implemented by aspects of a communication device 105, a server 135, and/or a care gap management engine 182 described with reference to FIG. 1. The program mechanics may include examples of aspects of any figure described herein.

The server 135 may identify member-level care opportunities for a set of members with diabetes. For example, the server 135 may assess a set of gaps-in-care (e.g., 71 care gaps) that members with diabetes may be eligible for, depending on their individual situation. An example of care gap opportunities associated with five (5) members is illustrated in Table 1 below:

TABLE 1

| | Members | | | | |
|---|---|---|---|---|---|
| | Alice | Bob | Charles | Daphne | Eleanor |
| Gap Category | F, 26 | M, 56 | M, 33 | F, 43 | F, 49 |
| Medication Optimization | | | | X | X |
| Medication Adherence | | | X | | X |
| SMBG | | | | | X |
| Screening | X | X | | | X |
| Predicted A1c | 7.9 | 10.4 | 6.6 | 9.0 | 12.1 |

Overall Average Predicted A1c: 7.5

The server 135 may estimate impact to a member's predicted A1c associated with closing any open gaps-in-care. For example, the server 135 (e.g., care gap management engine 182) may use a machine learning model(s) 184 (e.g., an A1c prediction model) described herein to accurately predict a member's future A1c value over millions of other diabetic members associated with a healthcare provider and/or an insurance provider. Using the A1c prediction model, the server 135 may estimate a non-causal heterogeneous impact to a member's predicted A1c associated with closing open gaps-in-care corresponding to the member. For example, the server 135 may predict the reduction in a member's A1c per each gap-in-care that is closed (e.g., behavior changed).

Aspects of the program mechanics may support a mean absolute error of A1c prediction of 0.5 A1c, with an R² of ca. 0.68-0.74. Accordingly, for example, aspects of the present disclosure support a mean absolute error meaning having an accuracy equal to (e.g., within a threshold value) that of a commercial glucometer in predicting A1c. For each member, we predict the impact to a member's predicted A1c associated with each gap. For example, in the example Table 1 illustrated above, a member Daphne had an open gap-in-care associated with ‘Medication optimization’.

According to example aspects of the present disclosure, the server 135 (e.g., care gap management engine 182, machine learning model(s) 184) may predict that closing the open gap-in-care associated with ‘Medication optimization’ would lower Daphne's predicted A1c by 1 unit, as illustrated in Table 2 below.

TABLE 2

| | Members | | | | |
|---|---|---|---|---|---|
| | Alice | Bob | Charles | Daphne | Eleanor |
| Gap Category | F, 26 | M, 56 | M, 33 | F, 43 | F, 49 |
| Medication Optimization | | | | −1.0 | −0.5 |
| Medication Adherence | | | −0.1 | | −1.1 |
| SMBG | | | | | −2.0 |
| Screening | −0.4 | −0.1 | | | −0.3 |
| Predicted A1c | 7.9 | 10.4 | 6.6 | 9.0 | 12.1 |

Overall Average Predicted A1c: 7.5

The server 135 may associate A1c impact to a cost impact (e.g., medical cost savings). For example, for each successfully closed gap, the server 135 may estimate the financial impact by multiplying the A1c impact of closing an open gap-in-care with (e.g., for a healthcare provider) a book-of-business-based, and literature backed, relationship between A1c reduction and the decrease in medical cost (e.g., adjusted for inflation).

In an example, for each member and each gap-in-care, the server 135 may cross-walk the A1c impact to medical cost savings, as illustrated in Table 3 below.

TABLE 3

| | Members | | | | |
|---|---|---|---|---|---|
| | Alice | Bob | Charles | Daphne | Eleanor |
| Gap Category | F, 26 | M, 56 | M, 33 | F, 43 | F, 49 |
| Medication Optimization | | | | $1400 | $700 |
| Medication Adherence | | | $140 | | $1540 |
| SMBG | | | | | $2800 |
| Screening | $560 | $140 | | | $420 |
| Predicted A1c | 7.9 | 10.4 | 6.6 | 9.0 | 12.1 |

The server 135 may constrain outreach (e.g., communications channel type, communications frequency, etc.) to a member by journey to optimize ROI, considering communication channel cost and efficacy. For example, the server 135 may generate a personalized prediction of the impact of closing gaps-in-care for an individual member with diabetes. In some aspects, the personalized prediction may implement a multi-channel outreach ROI optimization method.

That is, for example, aspects of the present disclosure support a targeted approach capable of (e.g., preferentially) targeting gaps-in-care associated with relatively higher-value members. In an example, aspects of the targeted approach may include ‘nudging’ the member through multiple communication channels with reference to the same gap-in-care. In another example, aspects of the targeted approach may include targeting gaps-in-care associated with relatively lower-value members by deploying more cost efficient channels (e.g., digital communication channels such as emails, SMS messages, etc.).

Table 4 below illustrates an example of a set of communication channels, corresponding probabilities of closing gaps-in-care, and corresponding costs. In some aspects, the server 135 may apply the corresponding probabilities and costs to facilitate ROI optimization.

TABLE 4

| Channel | Probability of Gap Closure | Cost of Outreach |
|---|---|---|
| Digital | 10% | $0.01 |
| SMS | 11% | $0.01 |
| Email | 10% | $0.01 |
| Direct Mail (Letter) | 4% | $1 |
| Pharmacist CPCO | 2.11% | $1.96 |
| Health Hub | 6.29% | $4.85 |
| Care Coordinator | 9.30% | $15.00 |

The server 135 (e.g., care gap management engine 182) may assign a member a journey using the information from Table 4. For example, for each member, the server 135 may prioritize a higher ranked or more/most valuable gap(s)-in-care associated with an intervention outreach strategy. In an example, the server 135 may assign a journey to a member based on a function of the value of the member's highest ranked gap-in-care, the cost of outreach, and the probability of closing the gap-in-care, for each communication channel.

Table 5 illustrates an example of journey assignment associated with relatively higher targeting of higher value gaps-in-care (e.g., by deploying a highest number of communication channels in a “High Touch” journey), relatively medium targeting of medium value gaps-in-care (e.g., by deploying, in a “Medium Touch” journey, relatively fewer communication channels compared to the “High Touch” journey), and relatively lower targeting of lower value gaps-in-care (e.g., by deploying, in a “Low Touch” journey, relatively fewer communication channels compared to the “Medium Touch” journey).

TABLE 5

| | Members | | | | |
|---|---|---|---|---|---|
| | Alice | Bob | Charles | Daphne | Eleanor |
| Gap Category | F, 26 | M, 56 | M, 33 | F, 43 | F, 49 |
| Medication Optimization | | | | $1400 | $700 |
| Medication Adherence | | | $140 | | $1540 |
| SMBG | | | | | $2800 |
| Screening | $560 | $140 | | | $420 |
| Predicted A1c | 7.9 | 10.4 | 6.6 | 9.0 | 12.1 |
| Journey | Medium | Medium | Low | High | High |

Referring to the example of Table 5, Daphne's highest value gap-in-care is ‘Medicine optimization’, which is worth $1400 per year in cost impact (e.g., medical cost savings). For example, the gap-in-care associated with Daphne may trigger (e.g., at the server 135, based on the cost impact compared to a threshold ROI) a “High Touch” journey for Daphne. In another example, Charles' highest value gap-in-care is ‘Medication Adherence’, which is worth $140 per year in cost impact. Accordingly, for example, the gap-in-care associated with Charles may trigger (e.g., at the server 135, based on the cost impact compared to a threshold ROI) a “Low Touch” journey.

Table 6 illustrates an example of communication channels implemented for each of the “High Touch”, “Medium Touch”, and “Low Touch” journeys. In the example of Table 6, proactive calls may be provided by a pharmacist, a healthcare provider, a health hub, and/or a care coordinator.

TABLE 6

| | | | | IVR | |
|---|---|---|---|---|---|
| | | | Direct Mail | (Automated | Proactive |
| Journey Type | Email | SMS | (Letter) | Call) | Call |
| High Touch | X | X | X | X | X |
| Medium Touch | X | X | X | X | |
| Low Touch | X | X | X | | |

### Journey Optimization

Aspects of the present disclosure support features for refining and/or optimizing the journeys described herein with respect to managing a care gap. The journey optimization may be implemented, for example, by the server 135 (e.g., using the care gap management engine 182). In some examples, the journey optimization may include features for identifying which care gap(s) to address and/or communication channels for addressing the identified care gap(s) (also referred to herein as new ‘gaps-to-address’). Aspects of the journey optimization may include features for selecting a new communication channel via which the server 135 may interact with a member in connection with closing (or at least partially closing) a new gap-in-care.

Examples of the journey optimization implementable by the server 135 may address problems of some systems with respect to improving management of medical conditions (e.g., improving diabetes management, etc.). For example, management of the medical conditions as described herein may include developing and refining a targeting model in association with accommodating a relatively large number of care gaps (e.g., 200+ care caps) of members of a healthcare provider. In some cases, a healthcare provider may have multiple different nurse channels, each with different capacity limits. Accordingly, for example, a healthcare provider could potentially have two, three, four, . . . , ten, or more channels to choose from to communicate care gap opportunities to members (or healthcare personnel) in association with achieving health improvements of the member.

Aspects of journey optimization described herein include (1) implementing many-to-many targeting (also referred to herein as many-to-many assignment or resource assignment), which may support determining how to pair communication channels and care gaps to drive maximum behavior change of a member. Journey optimization described herein includes features for (2) maintaining customizations as a healthcare management program expands (e.g., due to increased members, increased gaps-in-care, increased communication channels, etc.). In some cases, implementing journey optimization described herein, the server 135 may manage communication channel preferences at scale and at granular levels.

Journey optimization described herein may be implemented by the server 135 using any combination of computer programming languages capable of, for example, automating tasks, conducting data analysis, or the like. An example includes a general-purpose language (e.g., Python, etc.), but is not limited thereto. In some aspects, the server 135 may use a generalized python package leveraging linear and/or non-linear optimization to implement many-to-many targeting described herein. Additionally, or alternatively, the server 135 may apply linear optimization in combination with a rule-based targeting method. In an example, given a set of care gaps and associated values of the care gaps per member, the server 135 may implement journey optimization described herein to close (or at least partially close) a current gap-in-care and/or another gap-in-care.

In some cases, the server 135 (e.g., care gap management engine 182) may analyze a combination of inputs and generate outputs that support journey optimization described herein. Example inputs include, but are not limited to: care gaps available for each member, estimated value associated with each care gap, available communication channels (also referred to herein as “channels” or “intervention channels”) and their probabilities of successful engagement with the member, previously used communication channels, and any combination of associated constraints. Some example constraints include, but are not limited to: constraints about what gaps can or cannot be combined together, constraints associated with the capacity of a communication channel, constrains prohibiting reuse or overuse of a communication channel, per-unit costs associated with a communication channel, and combined costs associated with a communication channel (or group of communication channels). Some example constraints include overall cost constraints, either globally, by a plan sponsor (e.g., a healthcare provider, an insurance provider, etc.), by member, or other.

Examples of the outputs include, but are not limited to: identification of gaps-in-care to be “activated” for each member, identification of communication channels to be “activated” for addressing each gap-in-care, and overall cost and value associated with the gaps-in-care and/or communication channels to be “activated.” In an example, “activating” a gap-in-care may include selecting or targeting a gap-in-care to close. In some examples, “activating” a communication channel may include selecting the communication channel and providing communications to a member via the communication channel, in association with closing the gap-in-care.

Examples supportive of journey optimization for members according to some embodiments of the present disclosure are described herein. In an example, members Alice and Bob have the following data regarding their gaps-in-care and respective values (e.g., medical costs savings, etc.) associated with closing those gaps-in-care:

| Member | Gap ID | Value |
|---|---|---|
| Alice | 74 | $30 |
| Alice | 120 | $30 |
| Bob | 74 | $50 |

In the example, email communication is the only communication channel/intervention available for the members and has a 10% probability of success.

Scenario 1: The server 135 may identify that, if no gaps-in-care can be grouped together, and the capacity of the communication channel is 1 email (e.g., there is only capacity to send 1 email total), the server 135 may select the following as a solution:

| Member | Gap ID | Value | Send Email? |
|---|---|---|---|
| Alice | 74 | $30 | No |
| Alice | 120 | $30 | No |
| Bob | 74 | $50 | Yes |

Optimal solution for scenario 1:
Send email to Bob regarding Gap Id 74.
Total expected value $50.

Scenario 2: The server 135 may identify that, if multiple gaps can be grouped together for a member journey (e.g., if gaps 74 and 120 can be grouped together in the same journey), and the capacity of the communication channel is 1 email, the server 135 may select the following as a solution:

| Member | Gap ID | Value | Send Email? |
|---|---|---|---|
| Alice | 74 | $30 | Yes |
| Alice | 120 | $30 | |
| Bob | 74 | $50 | No |

Optimal solution for scenario 1:
Send email to Alice regarding Gap IDs 74 and 120.
Total expected value $60.

### Example Solution Framework

In some aspects, the server 135 may calculate the expected value using classic and standard mathematical optimization techniques. For example, the server 135 may apply a combination of classic and standard mathematical optimization techniques to find a global, overall solution that satisfies all necessary constraints.

In an example, the server 135 may calculate the expected value based on the following, but is not limited thereto:
(i) Members, indexed by i from 1 . . . N
(ii) Gaps (also referred to herein as an opportunity to close the gap), indexed by g from 1 . . . Gᵢ, where each member has a different set of gaps.
(iii) Channels, indexed by k from 1 . . . K

In an example, the server 135 may calculate the expected value using the following equation:

Expected Value=ΣᵢΣ_{g}Σₖpₖ·v_{ig}·x_{igk},

The equation includes the following example variables:
(i) probability (pₖ) of success for a channel k
(ii) value (v_{ig}) of closing gap g for member i
(iii) decision variable (x_{igk}). The server 135 may set x_{igk} to 0 or 1 for each member. For example, for a member i, the server 135 may set x_{igk} to 0 or 1 depending on whether the server 135 decides to utilize channel k in association with addressing gap g for the member i.

In some aspects, the server 135 may select a channel k for communicating to a member, based on the probability (pₖ) of success for channel k and value (v_{ig}) of closing gap g for member i. In some cases, the server 135 may select the channel k based on whether the use of the channel k is less than or equal to a capacity cap (capₖ) of the channel k. Additionally, or alternatively, the server 135 may select the channel k based on whether a cost (costₖ) associated with using the channel k is less than or equal to a cost budget.

### Example Implementation Considerations

Gap Bundling: Aspects of the present disclosure include setting guidelines on what gap(s) can be bundled together or not. In some examples, the server 135 may implement gap bundling based on the guidelines as a pre-processing step, prior to journey optimization described herein.

Gap/channel exclusions: Aspects of the present disclosure support a gap/channel exclusion, in which the server 135 may implement rule-based gap selection and/or rule-based channel selection. In an example, the server 135 may implement a rule that a channel k can never be used for a gap g. In an example implementation, the server 135 may add a constraint for all members i in which the decision variable x_{igk} is set to zero (in association with utilizing a specific channel k for a specific gap g).

Member-specific constraints: Aspects of the present disclosure support member-specific constraints such as, for example, permissions. The server 135 may implement permissions-based care gap management, channel selection, and journey optimization applicable to situations where certain members, based on attribute, have certain channels disabled (e.g., turned off, cut off) for some/all gaps. In an example, based on attributes of a member (e.g., permissions provided by a member), the server 135 may refrain from using one or more such communication channels when communicating with the member regarding one or more gaps. In an example implementation, a member may have email permissions set to “off”, and the server 135 may refrain from communicating with the member via email with respect to care gap management.

Channel constraints: Aspects of the present disclosure support channel-specific constraints. The server 135 may provide communications to a member (and/or healthcare provider) via a channel k, in accordance with a specific limit Lₖ on the quantity of outreaches that can occur on channel k. For example, the server 135 may utilize the following equation:

$${\sum }_{i} {\sum }_{g} {x}_{i g k} \leq {L}_{k}$$

In an example implementation, the server 135 may set the decision variable x_{igk} (in association with a member i, a specific channel k, and a specific gap g) such that the quantity of communications via the channel k does not exceed the limit Lₖ.

Cost Budget: Aspects of the present disclosure support cost(k) constraints in association with the utilization of a specific channel k. The server 135 may provide communications to a member i (and/or healthcare provider) via a channel k and in association with a gap g, such that such the cost(k) of the communications does not a global cost cap of C. For example, the server 135 may utilize the following equation:

$${\sum }_{i} {\sum }_{g} c o s t ( k ) \cdot {x}_{i g k} \leq C$$

### Example Components of Journey Optimization

Examples of components of journey optimization are described herein. The server 135 may incorporate any combination of the components in association with implementing care gap management and journey optimization described herein.

Channel Permissions: For each communication channel, each member has a permissions flag having a value of 0 or 1. A communication channel is “off” for all gaps for a member if the value of the permissions flag is set to 0. In some aspects, channel permissions represents whether it is viable to reach a member through a specific channel. In an example, the server 135 (care gap management engine 182, campaign manager 515, etc.) may determine whether any channel permissions exist for a member, before associating the management of care gaps of the member to a communication channel. Otherwise, CPL will drop them before sending to downstream channels and cause discrepancies in the process of data transition.

Exclusions and Suppressions: The server 135 may incorporate exclusions and suppressions, in combination with channel permissions, with respect to care gap management and journey optimization. For example, the server 135 may incorporate exclusions and/or suppressions according to level (e.g., member level, business level, etc.), group (or group type), and/or communication channel.

In an example implementation, in developing a care gap management plan for a group of members (e.g., a diabetes cohort) or a subgroup of those members, the server 135 may suppress one or more care gaps from consideration when targeting members of the group. For example, a group of members may include diabetic members with hypertension, for which a healthcare provider intends to introduce an antihypertensive lifestyle. In some cases, the server 135 may develop antihypertensive medication optimization care gaps that are more specific in comparison to antihypertensive optimization care gaps deemed by the server 135 as relatively more general. The server 135, when providing care gap management in association with closing the more specific antihypertensive medication optimization care gaps, may suppress the more general antihypertensive optimization care gaps, which may thereby prevent duplicative outreach.

In another example implementation, the server 135 may suppress care gaps from being sent to certain channels based on one or more criteria. For example, the server 135 may suppress care gaps from being sent to pharmacy panels and clinics for all members of a diabetes cohort.

Channel identification and journey structure: Aspects of the present disclosure support multiple groups of channels, each acting differently. For example, the system 100 described herein may support a) direct communication (e.g., using any combination of communication channels described herein such as email, SMS, direct mail (letter), etc.), b) proactive calls, and c) reactive communication (e.g., using any combination of communication channels described herein). The server 135 may provide communications to a member and/or healthcare provider via any of the channels, based on a structure associated with care gap management and journey optimization as determined using the techniques described herein.

All gaps and potential groupings of the gaps: The system 100 may support multiple categories (e.g., five categories, etc.) for gaps-in-care. The server 135 may group gaps-in-care together based on one or more criteria. For example, the server 135 may group together any gaps-in-care that share the same call to action.

Channel constraints: The system 100 may support care gap management and journey optimization in view of multiple constraints. For example, in determining strategies associated with care gap management and journey optimization, the server 135 may evaluate each potential communication channel in view of channel capacity and channel cost. For example, over a whole program (or for sub-segments), the server 135 may determine from the channel capacity of a given channel that the channel may be used at most N times (e.g., per member, per gap-in-care, etc.). In another example, the server 135 may determine from the channel cost of a given channel that each invocation of the channel (e.g., a communication using the channel) has a fixed cost.

Budget: The system 100 may support care gap management and journey optimization in view of an overall budget (e.g., a monetary amount) that may be utilized among channel selection. For example, as described herein, each channel may have an associated cost.

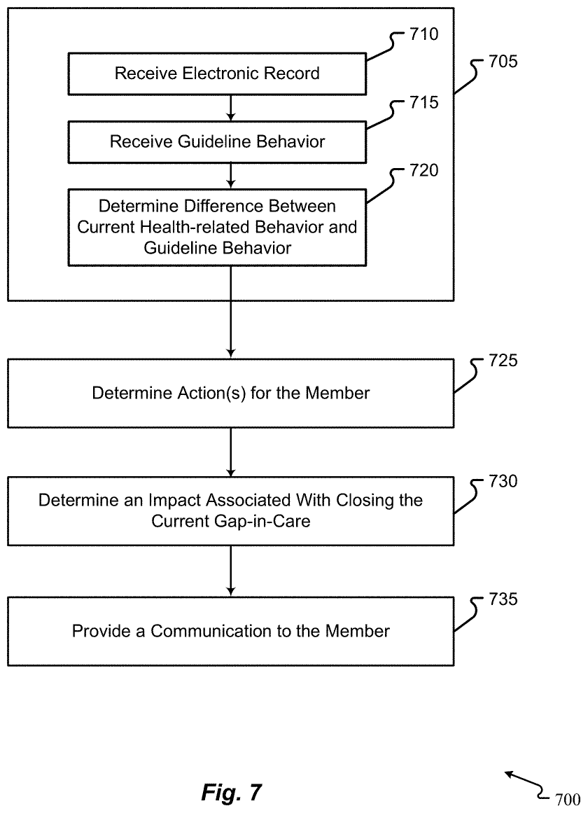

FIG. 7 illustrates an example of a process flow 700 that supports tracking chronic conditions in accordance with aspects of the present disclosure. For example, 700 may support observing member behavior and managing a care gap associated with the member. In some examples, process flow 700 may implement aspects of server 135 or a care gap management engine 182 described with reference to FIG. 1.

In the following description of the process flow 700, the operations may be performed in a different order than the order shown, or the operations may be performed in different orders or at different times. Certain operations may also be left out of the process flow 700, or other operations may be added to the process flow 700.

It is to be understood that while a server 135 is described as performing a number of the operations of process flow 700, any device (e.g., another server 135, a combination of servers 135, a communication device 105, a combination of a server 135 and a communication device 105) may perform the operations shown.

At 705, the server 135 may determine a current gap-in-care for a member. Example aspects associated with determining the current gap-in-care at 705 are described below with reference to 710 through 720.

At 710, the server 135 may receive an electronic record associated with the member that describes a current health-related behavior of the member.

In some aspects, the electronic record associated with the member may include claims-based electronic data. For example, the electronic record further may include electronic medical record (EMR) data. In another example, the claims-based electronic data may include data describing at least one insurance medical claim, pharmacy claim, and/or insurance claim made by at least one of the member and a provider. Accordingly, for example, the claims-based electronic data may come from providers or payers, and claims included in the claims-based electronic data may be of various types (e.g., medical, pharmacy, etc.).

In some other aspects, the electronic record associated with the member may include device data obtained from at least one device associated with the member. For example, the device data may include at least one of gyroscopic data, accelerometer data, beacon data, glucose readings, heart rate data, blood pressure data, blood oxygen data, temperature data, kinetics data, location data, motion data, a device identifier, and a timestamp.

In some aspects, the electronic record may include an image of the member. For example, the electronic record may include imaging data based on which the server 135 (e.g., the care gap management engine 182) may track targeted biomarkers. For example, the server 135 may track X-ray records of a member over time (e.g., in associated with assisting reduced healing times for a member).

In accordance with aspects of the present disclosure, the device data may be provided continuously, semi-continuously, periodically, and/or based on a trigger condition by the communication device 105 (e.g., a smart watch, a wearable monitor, a smartphone carried by a user, etc.) around monitored parameters such as heartbeat, blood pressure, etc. In some aspects, the device data may be referred to as “environmental data” associated with a user, which may be representative of aspects of environmental factors (e.g., lifestyle, socioeconomic factors, details about the environment, etc.) associated with a member.

Accordingly, for example, the electronic record may provide insurance claim information and/or generic behaviors of member behavior (e.g., behavior common to a set of members). In some cases, the electronic record may provide device data such as wearable-device data, glucose readings, heart rate, body temperature, “invisible” data (e.g., device related information associated with a member, such as Bluetooth beacon information), self-reporting monitored data (e.g., provided by self-reporting glucometers such as continuous glucose monitors (CGMs) that report kinetics).

In some aspects, the electronic record may include genetic data associated with a member. In some other aspects, the electronic record may include notes/documentation that is recorded in a universal and/or systematic format (e.g., subjective, objective, assessment, and plan (SOAP) notes/documentation) among medical providers, insurers, etc. In some examples, the electronic record may include non-claim adjudicated diagnoses (e.g., diagnoses that have not been evaluated by an insurance provider with respect to payment of benefits).

At 715, the server 135 may receive guideline behavior for the member supported by a professional clinical recommendation. In some examples, the guideline behavior for the member supported by the professional clinical recommendation may include guidance based on at least one of medical history, demographics, social indices, biomarkers, behavior data, engagement data, historical gap-in-care data, and a machine learning model-derived output (e.g., a risk-based model probability).

In some aspects, the gap-in-care described herein may be defined by a difference between guideline behavior associated with what a member should be doing, as defined by clinical guidelines and expert clinical opinion (e.g., professional guidelines surrounding preventative screenings and close follow-up and monitoring with providers) and current health related behavior associated with what the member is actually doing, which may be defined by static or longitudinal observables in the medical history of the member and supporting data.

At 720, the server 135 may determine a difference between the current health-related behavior of the member and the guideline behavior for the member. In some aspects, the difference defines, at least in part, the current gap-in-care for the member. For example, the guideline behavior may be driven by established professional recommendations and medical guidelines.

At 725, the server 135 may determine, for the current gap-in-care for the member, one or more actions for the member that, if followed, are capable of at least partially closing the current gap-in-care within a clinically-defined period of time for the member. Accordingly, for example, aspects of the present disclosure may support completion of gap closure within a clinically-defined window of time. In some aspects, the window may be set (e.g., by an evaluation entity such as, for example, an insurance provider and/or a medical provider) prior to outreach for measurement and evaluation purposes.

At 730, the server 135 may determine, for the one or more actions, an impact associated with at least partially closing the current gap-in-care for the member according to the one or more actions.

In some aspects, the impact may include a clinical impact. In an example, the clinical impact may be measured by a health biomarker. In some examples, the health biomarker may include at least one of HbA1c, blood pressure, and health complications. In some examples, the health complications may include at least one of a stroke, myocardial infraction, in-member admission, and emergency room admission.

In some other aspects, the impact may include a cost impact.

In some examples, the determined impact may be adjusted (e.g., by the server 135) by a baseline biomarker of the member. In some aspects, the determined impact may be adjusted (e.g., by the server 135) by a degree of member management of the member's condition.

In some aspects, the impact associated with at least partially closing the current gap-in-care for the member may include at least partially closing one or more additional current gaps-in-care for the member.

Accordingly, for example, aspects of the present disclosure may support predicting an ROI associated with closing (e.g., partially, fully, etc.) the current gap-in-care.

At 735, the server 135 may provide a communication to the member that describes the one or more actions for the member.

In some aspects, the communication may be delivered via a selected communication channel. In an example, the selected communication channel may be selected (e.g., by the server 135) based on a probability of closing the current gap-in-care. In some examples, the selected communication channel may include at least one of email, direct mail, SMS, and an automated outbound calling campaign. In some other examples, the communication channel may include interactive voice response (IVR) communications or a live phone call.

In some aspects, the communication channel may be delivered using one or more techniques associated with (e.g., grounded in) behavioral economics. For example, the communication channel may be delivered using techniques modeled (e.g., using behavioral models, where the behavioral models may be a machine learning model 184 described herein) in association with insights from psychology, neuroscience, and/or microeconomic theory. In some cases, the behavioral models may be generated and/or trained (e.g., by the server 135) based on effects of psychological, cognitive, emotional, cultural, and/or social factors on the decisions of individuals (e.g., members) and institutions (e.g., medical providers, insurance providers, etc.) and variances associated with those decisions.

Accordingly, for example, aspects of the process flow 700 may support predicting the future of a member and acting on the prediction (e.g., provide a communication to the member that describes the one or more actions for the member for closing the current gap-in-care within a period of time). The predicted future may include information such as health biomarkers (e.g., HbA1c, blood pressure, etc.), complications (e.g., stroke, myocardial infarction, IP/ER admissions rates), and cost impact (e.g., total cost of care or disease-related spending), in which the information is conditional on addressing (e.g., closing or managing) the current gap-in-care and/or other gaps-in-care.

As described with reference to the process flow 700, aspects of the present disclosure may support the prediction of an impact to the predicted health of a member (e.g., a clinical impact) or a future cost (e.g., a cost impact). In an example, the server 135 (e.g., care gap management engine 182) may predict the impact using a model explainer approach including a combination of game theory and machine learning prediction. In another example, the server 135 may predict the impact by causal inference (e.g., heterogenous treatment effect estimate.

In some aspects, the predicted impact may be adjusted by baseline biomarkers associated with a member and/or degree of management of a condition of the member. For example, the treatment effect in influencing a given biomarker may be correlated with the baseline value of a corresponding laboratory corresponding.

In another aspect, the predicted impact associated with a member may be adjusted (e.g., by the server 135, the care gap management engine 182) by the incremental contribution to predicted impact. In some cases, the predicted impact may be decreased (e.g., discounted) for members with multiple gaps in care.

In some other aspects, the predicted impact associated with a member may be adjusted (e.g., by the server 135, the care gap management engine 182) based on the ability of the intervention (e.g., treatment effect of actions determined for the member) to address the targeted gap in care.

In some aspects, the predicted impact associated with a member may be adjusted (e.g., by the server 135, the care gap management engine 182) by an assessment of the member's willingness and proclivity to engage and close their gaps via certain channels, as well as an understanding of any barriers contributing to health inertia (e.g., convenience, cost, access to care) for each member.

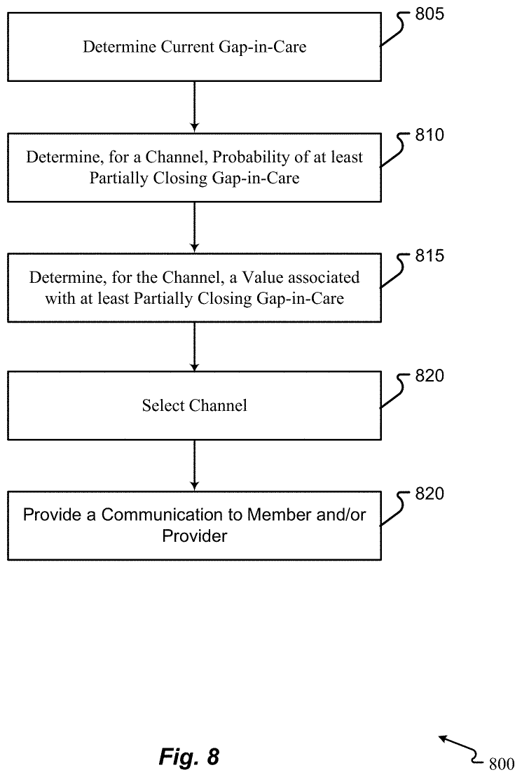

FIG. 8 illustrates an example of a process flow 800 that supports tracking chronic conditions in accordance with aspects of the present disclosure. For example, 800 may support channel selection for managing a care gap associated with a member. In some examples, process flow 800 may implement aspects of server 135 or a care gap management engine 182 described with reference to FIG. 1.

In the following description of the process flow 800, the operations may be performed in a different order than the order shown, or the operations may be performed in different orders or at different times. Certain operations may also be left out of the process flow 800, or other operations may be added to the process flow 800.

It is to be understood that while a server 135 is described as performing a number of the operations of process flow 800, any device (e.g., another server 135, a combination of servers 135, a communication device 105, a combination of a server 135 and a communication device 105) may perform the operations shown.

At 805, the server 135 may determine a current gap-in-care for the member. In some aspects, the current gap-in-care for the member may be defined as a difference between a current health-related behavior of the member and a guideline behavior for the member;

At 810, the server 135 may determine, for a channel (e.g., a communications channel such as email, direct mail, short message service (SMS), IVR, etc.), a probability of at least partially closing the current gap-in-care within a clinically-defined period of time for the member.

At 815, the server 135 may determine, for the channel, a value (e.g., clinical impact, cost impact) associated with at least partially closing the current gap-in-care for the member.

At 820, the server 135 may select the channel from among a plurality of candidate channels based on a combination of the determined probability and the determined value associated with at least partially closing the current gap-in-care for the member.

In some aspects, the channel may be selected (e.g., by the server 135) based, at least in part, on one or more of the following: an average closure rate measured through a randomized control trial experimentation; a predicted average closure rate, regressed post-hoc using a causal inferential approach; and a predicted closure rate for the member, derived from a machine learning model.

In some aspects, the channel may be selected based, at least in part, on one or more permissions associated with providing communications to the member via one or more channels. In some other aspects, the channel may be selected based, at least in part, on a capacity of the channel in association with providing communications to the member or the provider. In some other aspects, the channel may be selected based, at least in part, on a cost associated with providing the communication using the channel. In some other aspects, the channel may be selected based, at least in part, on a set of rules associated with providing communications to the member and the provider.

In some aspects, the selected channel may include at least one of: a selected pharmacist; a health hub; a clinic; a direct communication (e.g., via email, direct mail, SMS, IVR, etc.); a digital application (e.g., a mobile application on a communication device 105); a trained team outreach (e.g., via telephonic and/or video communications); an in-home assessment (e.g., by a certified care provider such as a nurse or medical technician); and a provider outreach (e.g., by a medical provider, an insurance provider, etc.).

At 825, the server 135 may provide a communication to at least one of the member and the provider via the selected channel that describes one or more actions for the member to take in connection with at least partially closing the current gap-in-care for the member and an impact associated with taking the one or more actions.

In some aspects, the one or more actions may include (but is not limited to) at least one of the following: instructing the member to talk to a care provider; instructing the care provider to talk to the member; instructing the member to talk to a pharmacist; instructing the member to self-monitor a health indicator; instructing the member on lifestyle management; instructing the member to obtain a medical screening; instructing the member for medical adherence; querying the member to achieve a medication optimization; and a digital outreach.

Accordingly, for example, aspects of the process flow 800 may support predicting an expected value (e.g., a clinical impact, a cost impact such as an ROI value, etc.) able to be captured by closing one or more gaps in care for a given member or set of members, through a given action or set of actions, executed through a given channel or set of channels.

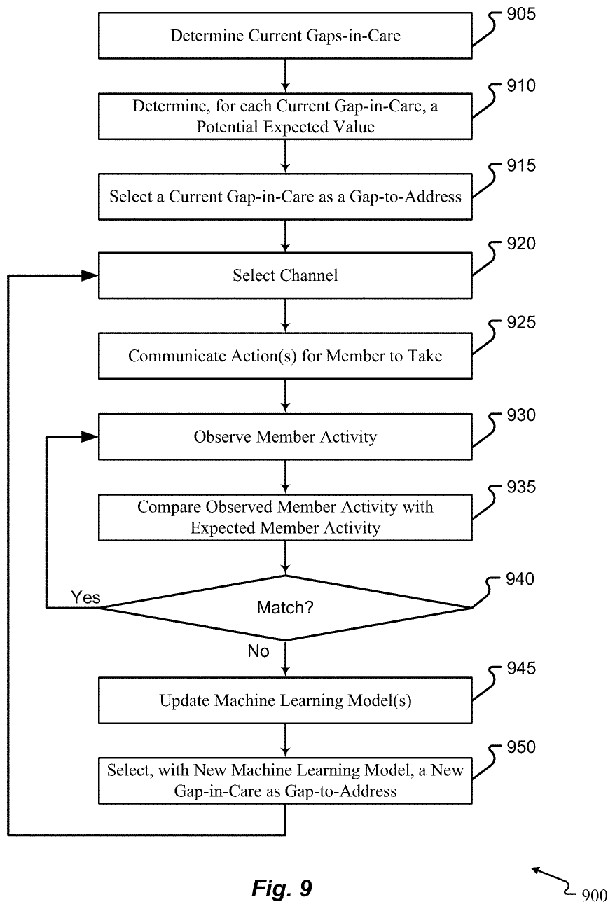

FIG. 9 illustrates an example of a process flow 900 that supports tracking chronic conditions in accordance with aspects of the present disclosure. For example, 900 may support managing care gaps associated with a member. In some examples, process flow 900 may implement aspects of server 135 or a care gap management engine 182 described with reference to FIG. 1.

In the following description of the process flow 900, the operations may be performed in a different order than the order shown, or the operations may be performed in different orders or at different times. Certain operations may also be left out of the process flow 900, or other operations may be added to the process flow 900.

It is to be understood that while a server 135 is described as performing a number of the operations of process flow 900, any device (e.g., another server 135, a combination of servers 135, a communication device 105, a combination of a server 135 and a communication device 105) may perform the operations shown.

At 905, the server 135 may determine a plurality of current gaps-in-care for the member. In an example, each of the plurality of current gaps-in-care for the member may be defined as a difference between a current health-related behavior of the member and a guideline behavior for the member.

At 910, the server 135 may determine for each of the plurality of current gaps-in-care for the member a potential expected value to be captured by reducing the difference. In an example, the potential expected value may be an ROI value.

At 915, the server 135 may select a current gap-in-care from among the plurality of gaps-in-care as a gap-to-address. In some aspects, the current gap-in-care may be selected based on having a higher potential value as compared to at least one other gap-in-care among the plurality of gaps-in-care.

In an example, at 915, a first machine learning model (e.g., a machine learning model 184 described herein) may be used to select the current gap-in-care, from among the plurality of gaps-in-care, as the gap-to-address.

At 920, the server 135 may select a channel to provide a highest probability of achieving the higher potential value. In some aspects, the server 135 may select the channel in association with closing the current gap-in-care (e.g., as described with reference to process flow 700 and process flow 800).

In an example, at 920, a second machine learning model (e.g., another machine learning model 184 described herein) different from the first machine learning model may be used to select the channel.

At 925, the server 135 may communicate one or more actions for the member to take in connection with at least partially closing the gap-in-care. In some examples, the one or more actions may be communicated to the member via a channel selected (e.g., by the server 135) to provide a highest probability of achieving the higher potential value.

In some aspects, the current gap-in-care may be selected (e.g., by the server 135, by the first machine learning model) with an expectation that the one or more actions will simultaneously at least partially close an additional gap-in-care from among the plurality of gaps-in-care.

In some aspects, the current gap-in-care and the selected channel may be selected (e.g., by the server 135, by the first machine learning model) with reference to a budget that limits an amount of resources available to close the current gap-in-care as well as other gaps-in-care of other members.

At 930, the server 135 may observe member activity in response to the member receiving the one or more actions via the selected channel.

In some aspects, the member activity may be observed by collecting (e.g., by the server 135, by the communication device 105, etc.) one or more electronic records of the member after the one or more actions are communicated to the member via the channel selected to provide the highest probability of achieving the higher potential value.

At 935, the server 135 may compare the observed member activity with an expected member activity. In an example, the expected member activity may include the one or more actions communicated to the member via the channel.

At 940, the server 135 may determine whether the observed member activity matches the expected member activity. For example, the server 135 may determine whether the member has performed (e.g., has completed) at least some of the one or more actions. In an example, at 940, the server 135 may determine that the observed member activity does not match the expected member activity.

At 945, in response to determining that the observed member activity does not match the expected member activity, the server 135 may update at least one machine learning model used to select the current gap-in-care with a new machine learning model. Additionally, or alternatively, at 945, in response to determining that the observed member activity does match the expected member activity, the server 135 may return to 930.

At 950, the server 135 may select, with the new machine learning model, a new gap-in-care from among the plurality of gaps-in-care as a new gap-to-address.

In some aspects, the server 135 may select (e.g., at 920) a new channel to provide the member with one or more actions for the member to take in connection with at least partially closing the new gap-in-care. In some aspects, the new channel may be different from the channel (e.g., as previously selected at 920).

In an example, the channel is selected by the at least one machine learning model; and the new channel is selected by the new machine learning model. For example, the server 135 may select a channel based on an output (e.g., an indication of a channel) by the at least one machine learning model, and the server 135 may further select a new channel based on an output (e.g., an indication of the new channel) by the new machine learning model.

In another example, the new channel is selected using linear optimization.

In another example, the communicating of the one or more actions via the channel and the selecting of the new channel is based, at least in part, on rule-based targeting.

Aspects of the process flow 900 described herein may support using machine learning (e.g., based on ROI) when data is unknown, followed by collecting data, observing the data, and adjusting the data (e.g., using reinforcement learning). For example, a first decision (e.g., a selection at 915 of a current gap-in-care as a gap-to-address) by the server 135 may be based on cost of a selected channel and a cost of closing the gap-in-care with an available amount of resources, and a subsequent decision (e.g., a selection at 945 of a new gap-in-care as a new-gap-to-address) by the server 135 may be based on data resulting from actions followed by a member with respect to the first decision (e.g., observed member activity not matching expected member activity).

In some aspects, the system 100 may use members (e.g., people) as the adversarial network, and based on how the members perform, the server 135 may generate and/or train an initial member-level model (e.g., based on ROI), followed by updating and/or retraining the member-level model based on observations and over time.

Accordingly, for example, aspects of the process flow 900 may support techniques for optimizing value (e.g., clinical impact, cost impact, etc.), ROI (e.g., for an insurance provider, a medical provider, a member, etc.), and delivery (e.g., communications via a channel).

Example aspects of the present disclosure as described herein may include predicting the potential expected value (e.g., a clinical impact, a cost impact, etc. predicted by the server 135 or the care gap management engine 182) able to be captured by closing one or more gaps in care for a given member or set of members. In some examples, the probability of success in capturing the expected value may include commercial factors (e.g., enrollment, engagement, delivery/outreach method, delivery success, etc.).

Some other aspects of the present disclosure may include determining (for individual members, the complete population (e.g., all members), any subset thereof, or any combination thereof) the set of channels that are to be utilized for each open gap-in-care given the expected value (e.g., a clinical impact, a cost impact, etc. predicted by the server 135 or the care gap management engine 182). Aspects of the determination of the set of channels may include an optimized determination that incorporates operational, financial, and other factors, including, but not limited to: outreach constraints, multiple-program overlap constraints (e.g., if a member is pregnant, focusing on pregnancy and refraining from capturing diabetic values), clinical-guidance constraints, and cost/budget constraints. Additionally or alternative to optimizing the prediction of the potential expected value (e.g., a clinical impact, a cost impact, etc.), aspects of the present disclosure may support optimizing the prediction of associated derivatives and related quantities (e.g., ROI).

Accordingly, for example, aspects of the present disclosure may support improved selection of potential value-add actions for a member. For example, for cases in which the ability (e.g., processing ability), capacity, and/or money to “do it all” is finite, the techniques described herein support optimization for selecting actions which, if followed by a member, may maximize overall value/ROI, given known constraints (e.g., ability, capacity, money, etc.).

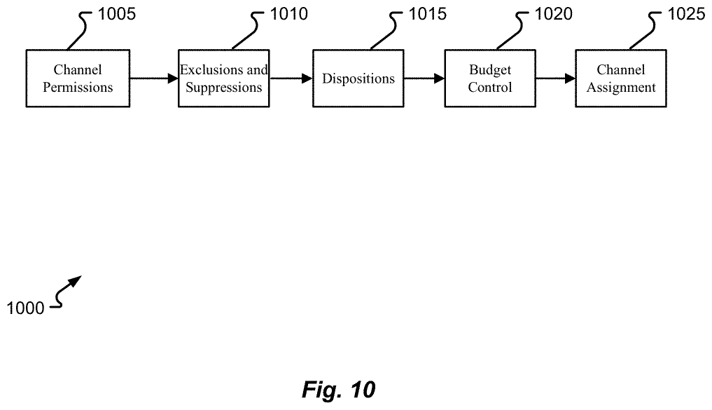

FIG. 10 illustrates an example operational flow 1000 that supports journey optimization deployment according to aspects of the present disclosure.

The operational flow 1000 may be based on the following example features and functionalities. The operational flow 1000, when implemented by the system 100, may achieve (1) many-to-many targeting while (2) maintaining customizations.

1. Channel Permissions (at 1005), examples of which are described above.

2. Exclusions and Suppressions (at 1010), examples of which are described above.

3. Dispositions (at 1015): Journey optimization may include the incorporation of dispositions delay logic into a targeting model. For example, the server 135 may categorize member dispositions acquired via channel×member interaction as negative/positive/neutral feedback. In an example, the server 135 may categorize a member request to opt out as negative, categorize an instance in which a member schedules an appointment at a local channel as positive, and categorize an undelivered communication as neutral. In some cases, corresponding to each care gap, the server 135 may apply a different impact for each category of dispositions.

In an example, positive, negative, and neutral dispositions may trigger respective delay periods (e.g., of X months, etc.). In some examples, in response to a positive/negative/neutral disposition with respect to a care gap, the server 135 may refrain (e.g., for a delay period corresponding to the disposition) from implementing an additional member outreach associated with the care gap, which may achieve improved member experiences.

4. Budget Control (at 1020): The system 100 may support a targeting model that assigns care gaps to channels based on respective values associated with closing the care gaps. For example, the targeting model may assign the care gaps to one or more specific channels if a) respective values associated with closing the care gaps are valuable (e.g., greater than or equal to a threshold) and b) appropriate to utilize the specific channels.

In an example of budget control, the server 135 may enable a total budget constraint or threshold associated with implementing member outreach. The total budget constraint or threshold may be, for example, a monthly intervention outreach budget. In an example, the total budget is accumulated based on member level monthly outreach budget and total identified diabetes members of the time.

The server 135 may implement member outreach while maintaining the costs associated with the outreaches below the total budget constraint. For example, the server 135 may calculate the costs based on a total quantity of identified diabetes members (diabetics) and the cost per identified diabetic (per month).

5. Channel assignment (at 1025): The server 135 may implements other common considerations (e.g., objectives, nurse channel capacity, conflicting channels, etc.) as constraints or parameters associated with assigning channels for care gap management.

### a. Example Objectives of Journey Optimizer

In some aspects, the server 135 may implement care gap management and journey optimization in which targeted care gap(s) and communication channels assigned to the targeted care gap(s) achieve a maximized value (e.g., total predicted medical cost saving realizable). In some alternative and/or additional aspects, the server 135 may target a maximized ROI.

In an example, the ROI=[(Value Associated with Managing a Care Gap)−(Total Channel Outreach Cost)]. In some aspects, the Value=SUM [predicted medical cost savings per closing a care gap×probability of closing a gap by a specific channel]. In some aspects, the Total Channel Outreach Cost=SUM [Number of channels selected for each gap×channel outreach cost at gap level].

### b. Nurse Channel Capacity

In some cases, the capacity of deliverable interventions may vary across different nurse channels. For example, for a given number of dedicated healthcare personnel (e.g., nurses, care managers, primary reviewers, etc.) there is a fixed quantity of members that the healthcare personnel can contact during a temporal period (e.g., one month).

In some aspects of journey optimization, the server 135 may apply the quantity of members as a constraint such that assigned opportunity load for managing care gaps of the members does not exceed the capacity of the healthcare personnel, while keeping the assigned opportunity load above a threshold (e.g., always filled).

In an example of the assigned opportunity load to nurse channel:

1) 90% of healthcare personnel capacity≤Number of members≤110% of healthcare personnel capacity

2) The value of each care gap assigned to nurse channel≥Expected cost of closing a gap by nurse channel. In an example, the cost of closing a gap by nurse channel=(nurse channel cost)/(probability of success).

### c. Conflicting Channels

The server 135 may specify that a given combination of channels (or delivery channel systems) cannot be co-assigned for member outreach with respect to a member and a temporal period (e.g., during each monthly outreach). Accordingly, for example, the server 135 may prevent or minimize repetitive communications (e.g., calls, emails, letters, SMS messages, etc.) from different people about the same topics such as managing a gap-in-care.

A number of implementations have been described. Nevertheless, it will be understood that additional modifications may be made without departing from the scope of the inventive concepts described herein, and, accordingly, other examples are within the scope of the following claims.

Additional details of the disclosure are in the following attachments, each of which is incorporated herein by this reference:

Exhibit A—Precision Diabetes Manuscript_Aug. 3, 2021

The exemplary systems and methods of this disclosure have been described in relation to examples of a communication device 105 and a server 135. However, to avoid unnecessarily obscuring the present disclosure, the preceding description omits a number of known structures and devices. This omission is not to be construed as a limitation of the scope of the claimed disclosure. Specific details are set forth to provide an understanding of the present disclosure. It should, however, be appreciated that the present disclosure may be practiced in a variety of ways beyond the specific detail set forth herein.

Furthermore, while the examples illustrated herein show the various components of the system collocated, certain components of the system can be located remotely, at distant portions of a distributed network, such as a LAN and/or the Internet, or within a dedicated system. Thus, it should be appreciated, that the components of the system can be combined into one or more devices, such as a server, communication device, or collocated on a particular node of a distributed network, such as an analog and/or digital telecommunications network, a packet-switched network, or a circuit-switched network. It will be appreciated from the preceding description, and for reasons of computational efficiency, that the components of the system can be arranged at any location within a distributed network of components without affecting the operation of the system.

Furthermore, it should be appreciated that the various links connecting the elements can be wired or wireless links, or any combination thereof, or any other known or later developed element(s) that is capable of supplying and/or communicating data to and from the connected elements. These wired or wireless links can also be secure links and may be capable of communicating encrypted information. Transmission media used as links, for example, can be any suitable carrier for electrical signals, including coaxial cables, copper wire, and fiber optics, and may take the form of acoustic or light waves, such as those generated during radio-wave and infra-red data communications.

While the flowcharts have been discussed and illustrated in relation to a particular sequence of events, it should be appreciated that changes, additions, and omissions to this sequence can occur without materially affecting the operation of the disclosed examples, configuration, and aspects.

A number of variations and modifications of the disclosure can be used. It would be possible to provide for some features of the disclosure without providing others.

In yet another example, the systems and methods of this disclosure can be implemented in conjunction with a special purpose computer, a programmed microprocessor or microcontroller and peripheral integrated circuit element(s), an ASIC or other integrated circuit, a digital signal processor, a hard-wired electronic or logic circuit such as discrete element circuit, a programmable logic device or gate array such as PLD, PLA, FPGA, PAL, special purpose computer, any comparable means, or the like. In general, any device(s) or means capable of implementing the methodology illustrated herein can be used to implement the various aspects of this disclosure. Exemplary hardware that can be used for the present disclosure includes computers, handheld devices, telephones (e.g., cellular, Internet enabled, digital, analog, hybrids, and others), and other hardware known in the art. Some of these devices include processors (e.g., a single or multiple microprocessors), memory, nonvolatile storage, input devices, and output devices. Furthermore, alternative software implementations including, but not limited to, distributed processing or component/object distributed processing, parallel processing, or virtual machine processing can also be constructed to implement the methods described herein.

In yet another examples, the disclosed methods may be readily implemented in conjunction with software using object or object-oriented software development environments that provide portable source code that can be used on a variety of computer or workstation platforms. Alternatively, the disclosed system may be implemented partially or fully in hardware using standard logic circuits or VLSI design. Whether software or hardware is used to implement the systems in accordance with this disclosure is dependent on the speed and/or efficiency requirements of the system, the particular function, and the particular software or hardware systems or microprocessor or microcomputer systems being utilized.

In yet another example, the disclosed methods may be partially implemented in software that can be stored on a storage medium, executed on programmed general-purpose computer with the cooperation of a controller and memory, a special purpose computer, a microprocessor, or the like. In these instances, the systems and methods of this disclosure can be implemented as a program embedded on a personal computer such as an applet, JAVA® or CGI script, as a resource residing on a server or computer workstation, as a routine embedded in a dedicated measurement system, system component, or the like. The system can also be implemented by physically incorporating the system and/or method into a software and/or hardware system.

Although the present disclosure describes components and functions implemented in the examples with reference to particular standards and protocols, the disclosure is not limited to such standards and protocols. Other similar standards and protocols not mentioned herein are in existence and are considered to be included in the present disclosure. Moreover, the standards and protocols mentioned herein and other similar standards and protocols not mentioned herein are periodically superseded by faster or more effective equivalents having essentially the same functions. Such replacement standards and protocols having the same functions are considered equivalents included in the present disclosure.

The present disclosure, in various examples, configurations, and aspects, includes components, methods, processes, systems and/or apparatus substantially as depicted and described herein, including various examples, subcombinations, and subsets thereof. Those of skill in the art will understand how to make and use the systems and methods disclosed herein after understanding the present disclosure. The present disclosure, in various examples, configurations, and aspects, includes providing devices and processes in the absence of items not depicted and/or described herein or in various examples, configurations, or aspects hereof, including in the absence of such items as may have been used in previous devices or processes, e.g., for improving performance, achieving ease, and/or reducing cost of implementation.

The foregoing discussion of the disclosure has been presented for purposes of illustration and description. The foregoing is not intended to limit the disclosure to the form or forms disclosed herein. In the foregoing Detailed Description for example, various features of the disclosure are grouped together in one or more examples, configurations, or aspects for the purpose of streamlining the disclosure. The features of the examples, configurations, or aspects of the disclosure may be combined in alternate examples, configurations, or aspects other than those discussed above. This method of disclosure is not to be interpreted as reflecting an intention that the claimed disclosure requires more features than are expressly recited in each claim. Rather, as the following claims reflect, inventive aspects lie in less than all features of a single foregoing disclosed example, configuration, or aspect. Thus, the following claims are hereby incorporated into this Detailed Description, with each claim standing on its own as a separate preferred example of the disclosure.

Moreover, though the description of the disclosure has included description of one or more examples, configurations, or aspects and certain variations and modifications, other variations, combinations, and modifications are within the scope of the disclosure, e.g., as may be within the skill and knowledge of those in the art, after understanding the present disclosure. It is intended to obtain rights, which include alternative examples, configurations, or aspects to the extent permitted, including alternate, interchangeable and/or equivalent structures, functions, ranges, or steps to those claimed, whether or not such alternate, interchangeable and/or equivalent structures, functions, ranges, or steps are disclosed herein, and without intending to publicly dedicate any patentable subject matter.

The phrases “at least one,” “one or more,” “or,” and “and/or” are open-ended expressions that are both conjunctive and disjunctive in operation. For example, each of the expressions “at least one of A, B and C,” “at least one of A, B, or C,” “one or more of A, B, and C,” “one or more of A, B, or C,” “A, B, and/or C,” and “A, B, or C” means A alone, B alone, C alone, A and B together, A and C together, B and C together, or A, B and C together.

The term “a” or “an” entity refers to one or more of that entity. As such, the terms “a” (or “an”), “one or more,” and “at least one” can be used interchangeably herein. It is also to be noted that the terms “comprising,” “including,” and “having” can be used interchangeably.

The term “automatic” and variations thereof, as used herein, refers to any process or operation, which is typically continuous or semi-continuous, done without material human input when the process or operation is performed. However, a process or operation can be automatic, even though performance of the process or operation uses material or immaterial human input, if the input is received before performance of the process or operation. Human input is deemed to be material if such input influences how the process or operation will be performed. Human input that consents to the performance of the process or operation is not deemed to be “material.”

Aspects of the present disclosure may take the form of an example that is entirely hardware, an example that is entirely software (including firmware, resident software, micro-code, etc.) or an example combining software and hardware aspects that may all generally be referred to herein as a “circuit,” “module,” or “system.” Any combination of one or more computer-readable medium(s) may be utilized. The computer-readable medium may be a computer-readable signal medium or a computer-readable storage medium.

A computer-readable storage medium may be, for example, but not limited to, an electronic, magnetic, optical, electromagnetic, infrared, or semiconductor system, apparatus, or device, or any suitable combination of the foregoing. More specific examples (a non-exhaustive list) of the computer-readable storage medium would include the following: an electrical connection having one or more wires, a portable computer diskette, a hard disk, a random access memory (RAM), a read-only memory (ROM), an erasable programmable read-only memory (EPROM or Flash memory), an optical fiber, a portable compact disc read-only memory (CD-ROM), an optical storage device, a magnetic storage device, or any suitable combination of the foregoing. In the context of this document, a computer-readable storage medium may be any tangible medium that can contain or store a program for use by or in connection with an instruction execution system, apparatus, or device.

A computer-readable signal medium may include a propagated data signal with computer-readable program code embodied therein, for example, in baseband or as part of a carrier wave. Such a propagated signal may take any of a variety of forms, including, but not limited to, electro-magnetic, optical, or any suitable combination thereof. A computer-readable signal medium may be any computer-readable medium that is not a computer-readable storage medium and that can communicate, propagate, or transport a program for use by or in connection with an instruction execution system, apparatus, or device. Program code embodied on a computer-readable medium may be transmitted using any appropriate medium, including, but not limited to, wireless, wireline, optical fiber cable, RF, etc., or any suitable combination of the foregoing.

The terms “determine,” “calculate,” “compute,” and variations thereof, as used herein, are used interchangeably and include any type of methodology, process, mathematical operation or technique.

What is claimed is:

1. A method of channel selection for managing a care gap associated with a member, the method comprising:
determining a current gap-in-care for the member, wherein the current gap-in-care for the member is defined as a difference between a current health-related behavior of the member and a guideline behavior for the member;
determining, for a channel, a probability of at least partially closing the current gap-in-care within a clinically-defined period of time for the member;
determining, for the channel, a value associated with at least partially closing the current gap-in-care for the member;
selecting the channel from among a plurality of candidate channels based on a combination of the determined probability and the determined value associated with at least partially closing the current gap-in-care for the member; and
providing a communication to at least one of the member and a provider via the selected channel that describes one or more actions for the member to take in connection with at least partially closing the current gap-in-care for the member and an impact associated with taking the one or more actions.

2. The method of claim 1, wherein the channel is selected based, at least in part, on one or more of the following:
an average closure rate measured through a randomized control trial experimentation;
a predicted average closure rate, regressed post-hoc using a causal inferential approach; and
a predicted closure rate for the member, derived from a machine learning model.

3. The method of claim 1, wherein the channel is selected based, at least in part, on one or more permissions associated with providing communications to the member via one or more channels.

4. The method of claim 1, wherein the channel is selected based, at least in part, on a capacity of the channel in association with providing communications to the member or the provider.

5. The method of claim 1, wherein the channel is selected based, at least in part, on a cost associated with providing the communication using the channel.

6. The method of claim 1, wherein the channel is selected based, at least in part, on a set of rules associated with providing communications to the member and the provider.

7. The method of claim 1, wherein the one or more actions comprise at least one of the following:
instructing the member to talk to a care provider;
instructing the care provider to talk to the member;
instructing the member to talk to a pharmacist;
instructing the member to self-monitor a health indicator;
instructing the member on lifestyle management;
instructing the member to obtain a medical screening;
instructing the member for medical adherence;
querying the member to achieve a medication optimization; and
a digital outreach.

8. The method of claim 1, wherein the selected channel comprises at least one of:
a selected pharmacist;
a health hub;
a clinic;
a direct communication;
a digital application;
a trained team outreach;
an in-home assessment; and
a provider outreach.

9. A method of managing care gaps associated with a member, the method comprising:
determining a plurality of current gaps-in-care for the member, wherein each of the plurality of current gaps-in-care for the member is defined as a difference between a current health-related behavior of the member and a guideline behavior for the member;
determining for each of the plurality of current gaps-in-care for the member a potential expected value to be captured by reducing the difference;
selecting a current gap-in-care from among the plurality of gaps-in-care as a gap-to-address, wherein the current gap-in-care is selected based on having a higher potential value as compared to at least one other gap-in-care among the plurality of gaps-in-care; and
communicating one or more actions for the member to take in connection with at least partially closing the gap-in-care, wherein the one or more actions are communicated to the member via a channel selected to provide a highest probability of achieving the higher potential value.

10. The method of claim 9, further comprising:
observing member activity in response to the member receiving the one or more actions via the selected channel;
comparing the observed member activity with an expected member activity;
determining that the observed member activity does not match the expected member activity;
in response to determining that the observed member activity does not match the expected member activity, updating at least one machine learning model used to select the current gap-in-care with a new machine learning model; and
selecting, with the new machine learning model, a new gap-in-care from among the plurality of gaps-in-care as a new gap-to-address.

11. The method of claim 10, further comprising:
selecting a new channel to provide the member with one or more actions for the member to take in connection with at least partially closing the new gap-in-care, wherein the new channel is different from the channel.

12. The method of claim 11, wherein:
the channel is selected by the at least one machine learning model; and
the new channel is selected by the new machine learning model.

13. The method of claim 11, wherein the new channel is selected using linear optimization.

14. The method of claim 11, the communicating of the one or more actions via the channel and the selecting of the new channel is based, at least in part, on rule-based targeting.

15. The method of claim 10, wherein the member activity is observed by collecting one or more electronic records of the member after the one or more actions are communicated to the member via the channel selected to provide the highest probability of achieving the higher potential value.

16. The method of claim 9, wherein a first machine learning model is used to select the current gap-in-care from among the plurality of gaps-in-care as the gap-to-address.

17. The method of claim 16, wherein a second machine learning model different from the first machine learning model is used to select the channel.

18. The method of claim 16, wherein the current gap-in-care is selected with an expectation that the one or more actions will simultaneously at least partially close the current gap-in-care and an additional gap-in-care from among the plurality of gaps-in-care.

19. The method of claim 9, wherein the current gap-in-care and the selected channel are selected with reference to a budget that limits an amount of resources available to close the current gap-in-care as well as other gaps-in-care of other members.

20. A system for managing care gaps associated with a member, comprising:
a processor; and
a memory coupled with the processor, wherein the memory stores data that, when executed by the processor, enables the processor to:
determine a plurality of current gaps-in-care for the member, wherein each of the plurality of current gaps-in-care for the member is defined as a difference between a current health-related behavior of the member and a guideline behavior for the member;
determine for each of the plurality of current gaps-in-care for the member a potential expected value to be captured by reducing the difference;
select a current gap-in-care from among the plurality of gaps-in-care as a gap-to-address, wherein the current gap-in-care is selected based on having a higher potential value as compared to at least one other gap-in-care among the plurality of gaps-in-care; and
communicate one or more actions for the member to take in connection with at least partially closing the gap-in-care, wherein the one or more actions are communicated to the member via a channel selected to provide a highest probability of achieving the higher potential value.
